(19) United States
(12) Patent Application Publication
(10) Pub. No.: US 2009/0247609 A1
(43) Pub. Date: Oct. 1, 2009

(54) SM-PROTEIN BASED SECRETION ENGINEERING

(76) Inventors: Hitto KAUFMANN, Ingelheim (DE); Eric BECKER, Ingelheim (DE); Lore FLORIN, Ingelheim (DE); Martin FUSSENEGGER, Maegenwil (CH); Ren-Wang PENG, Winterthur (CH); Joey M STUDTS, Ingelheim (DE)

Correspondence Address: MICHAEL P. MORRIS;BOEHRINGER INGELHEIM USA CORPORATION, 900 RIDGEBURY ROAD, P. O. BOX 368, RIDGEFIELD, CT, 06877-0368, US

(21) Appl. No.: 12/336,612

(22) Filed: Dec. 17, 2008

(30) Foreign Application Priority Data
Dec. 20, 2007 (EP) 07150254.6
Mar. 17, 2008 (EP) 08152829.1

(51) Int. Cl. A61K 31/7105; C12P 21/00; C12N 15/87; C12P 21/08; C12N 15/00; C12N 5/06; C07K 16/18; A61K 31/7088; C12Q 1/02

(52) U.S. Cl. 514/44 A; 435/69.1; 435/455; 435/69.6; 435/320.1; 435/326; 435/358; 530/387.3; 514/44 R; 435/29; 435/375

(57) ABSTRACT

The present invention concerns the field of cell culture technology. It describes a novel method for enhancing the secretory transport of proteins in eukaryotic cells by heterologous expression of Munc18c, Sly1 or other members of the SM protein family. This method is particularly useful for the generation of optimized host cell systems with enhanced production capacity for the expression and manufacture of recombinant protein products.

### RELATED APPLICATIONS

This application claims priority to European Application No. EP 07150254.6, filed Dec. 20, 2007 and to European Application No. EP 08152829.1, filed Mar. 17, 2008, each of which is hereby incorporated by reference in its entirety.

### BACKGROUND OF THE INVENTION

1. Technical Field

The invention concerns the field of cell culture technology. It concerns a method for producing proteins as well as a method to generate novel expression vectors and host cells for biopharmaceutical manufacturing. The invention further concerns pharmaceutical compositions and methods of treatment.

2. Background

The market for biopharmaceuticals for use in human therapy continues to grow at a high rate with 270 new biopharmaceuticals being evaluated in clinical studies and estimated sales of 30 billions in 2003. Biopharmaceuticals can be produced from various host cell systems, including bacterial cells, yeast cells, insect cells, plant cells and mammalian cells including human-derived cell lines. Currently, an increasing number of biopharmaceuticals is produced from eukaryotic cells due to their ability to correctly process and modify human proteins. Successful and high yield production of biopharmaceuticals from these cells is thus crucial and depends highly on the characteristics of the recombinant monoclonal cell line used in the process. Therefore, there is an urgent need to generate new host cell systems with improved properties and to establish methods to culture producer cell lines with high specific productivities as a basis for high yield processes. The yield of any biopharmaceutical production process depends largely on the amount of protein product that the producing cells secrete per time when grown under process conditions. Many complex biochemical intracellular processes are necessary to synthesize and secrete a therapeutic protein from a eukaryotic cell. All these steps such as transcription, RNA transport, translation, post-translational modification and protein transport are tightly regulated in the wild-type host cell line and will impact on the specific productivity of any producer cell line derived from this host.

In the past, most engineering approaches have focused on the molecular networks that drive processes such as transcription and translation to increase the yield of these steps in protein production. However, as for any multi-step production process, widening a bottle-neck during early steps of the process chain possibly creates bottle-necks further downstream, especially post translation in the secretory pathway. Up to a certain threshold, the specific productivity of a production cell has been reported to correlate linearly with the level of product gene transcription.

Further enhancement of product expression at the mRNA level, however, may lead to an overload of the protein synthesis, folding or transport machinery, resulting in intracellular accumulation of the protein product. Indeed, this can be frequently observed in current manufacturing processes. Therefore, the secretory transport machinery of the production cell line is an interesting target for novel host cell engineering strategies.

The first studies on engineering the intracellular transport of secreted therapeutic proteins were centered around the overexpression of molecular chaperones like binding protein BiP/GRP78 and protein disulfide isomerase (PDI). Chaperones are cellular proteins hosted within the endoplasmic reticulum (ER) and assist the folding and assembly of newly synthesised proteins. However in contrast to what could be expected, BiP overexpression in mammalian cells has been shown to reduce rather than increase the secretion of proteins it associates with, while overexpression of PDI in CHO cells yielded conflicting results with different protein products. A possible explanation for these surprising findings, that the increase of the cell's protein folding capacity creates a production bottle neck further downstream, is supported by a report describing ER to cis-Golgi transport problems for IFN-gamma production in a CHO cell line (Hooker et al., 1999).

In summary, there is a need for improving the secretory capacity of host cells for recombinant protein production. This might even become more important in combination with novel transcription-enhancing technologies and in high-titer processes in order to prevent post-translational bottle necks and intracellular accumulation of the protein product. However, at present, there are two major hurdles on the way to targeted manipulation of the secretory transport machinery: The still limited knowledge about the underlying regulatory mechanisms and the challenge to prevent shifting of bottle-necks to steps further downstream in the secretion process.

### SUMMARY OF THE INVENTION

The present invention describes a novel and surprising role of members of the Sec1/Munc18 (SM) protein family, particularly two members, namely Munc-18c and Sly1, in stimulating overall exocytosis by unitedly promoting several subsequent steps in the transport of secreted proteins to the cell surface and regulating the fusion of secretory vesicles with the plasma membrane. The present invention also provides a method to efficiently improve the production of proteins that are transported via the secretory pathway from eukaryotic cells. Furthermore, it describes the use of targeted manipulation of the secretory pathway for the treatment of diseases and inflammatory conditions.

Protein secretion is a complex multi-step mechanism: Proteins destined to be transported to the extracellular space or the outer plasma membrane are first co-translationally imported into the endoplasmic reticulum. From there, they are packed in lipid vesicles and transported to the Golgi apparatus and finally from the trans-Golgi network to the plasma membrane where they are released into the culture medium.

At each trafficking step, SNARE [soluble NSF (N-ethylmaleimide sensitive factor) attachment receptor] proteins from both vesicles and target membranes form trans-SNARE complexes that constitute the core machinery required for fusion to occur. To meet the physiological requirements in various situations, the SNARE-mediated fusion machinery must be spatially and temporally tunable in order for stimuli from both intracellular and extracellular sources to be integrated properly.

Sec1/Munc18 (SM) proteins seem to hold the key to regulating SNARE proteins. Two SM proteins, Sly1 and Munc18 (including a, b and c, three isoforms), are involved in vesicle fusion along the secretory pathway (ER-Golgi-plasma membranes). Sly1 is required for fusion to the Golgi apparatus of endoplasmic reticulum (ER)-derived COPII vesicles and Munc18 to the plasma membrane (PM) of secretory vesicles.

In the present invention, we analyzed the physiological impact of Sly1 and Munc18c on the secretory pathway and found for the first time that the two SM proteins unanimously stimulate overall exocytosis. The molecular mechanism of the activation role by Munc18c and Sly1 is likely conserved too. Based on the finding here, we pioneered an SM protein-based secretion engineering that results in enhanced secretion in mammalian cells. The SM protein-based secretion engineering represents a novel strategy of metabolic engineering and provides a new platform for the manufacturing of protein pharmaceuticals in industry.

The method described in the present invention is advantageous in several respects:

First, we demonstrate heterologous expression of either Munc-18c, Sly-1 or both proteins together to be a strategy to enhance recombinant protein production by increasing the secretory capacity of the host cell.

With respect to industrial application, the study opens the exiting perspective to bypass this bottle-neck by genetic engineering through introducing a transgene that exerts its action post-translationally in the secretory pathway. This appears of particular relevance as the use of the latest generation of highly efficient expression vectors might lead to an overload of the protein-folding, -modification and transport machinery within the producer cell line, thus reducing its theoretical maximum productivity. The heterologous introduction of secretion-enhancing proteins of the SM family, such as Munc18 and/or Sly1, can overcome this limitation.

Second, SM proteins are evolutionary conserved from yeast to men: In yeast, there are four SM proteins (Sec1p, Sly1p, Vps33p and Vps45p), three in drosophila (ROP, Sly1 and Vps33/carnation), six in worms (Unc-18 as well as 5 other genes according to genome sequence databases) as well as seven proteins in vertebrates (Munc18-1, -2 and -3, VPS45, VPS33-A and -B and Sly1). In light of the high degree of conservation across species, it seems very likely that SM proteins can be used to modulate secretion and cell-surface expression of proteins in all eukaryotic host cell species from yeast over worms and insect cells to mammalian systems.

Third, all members of the SM protein family show a high degree of sequence similarity over the entire sequence, suggesting that they should exhibit similar overall structures. Furthermore, loss-of-function mutations have been described for nine SM genes in four species, which all lead to severe impairment of vesicle trafficking and fusion, indicating that SM proteins should play similar and central roles in the process of vesicle transport and secretion. We therefore claim that the applicability of Munc18 and/or Sly1 for the purposes described in the present invention can be equally transferred to any other member of the SM protein family.

Fourth, by modulating the SNARE-mediated vesicle fusion machinery, members of the SM protein family are involved in all the different steps of vesicle trafficking from ER to Golgi, from Golgi to the plasma membrane and the final exocytotic fusion. Thus, heterologous expression of multiple SM proteins participating in subsequent steps of the secretory transport chain has the potential to yield an additive or even synergistic effect on overall exocytosis or cell-surface expression of transmembrane proteins. Moreover, simultaneous engineering of the ER as starting point of protein transport by heterologous co-expression of the transcription factor XBP-1 further increases this secretion enhancing effect.

As a fifth advantage, SM proteins also impact on the very last steps of the secretory pathway, namely vesicle transport to the plasma membrane, and thereby promote protein secretion without the risk of creating bottle-necks further downstream.

Taken together, the participation of SM proteins in all steps of vesicle-mediated protein transport from ER to Golgi and from the Golgi apparatus to the plasma membrane, make Munc18c, Sly1 and all other SM family proteins very attractive and promising targets for (multi-) genetic engineering approaches aiming to enhance the secretory capacity of eukaryotic cells.

The targeted engineering of the vesicle-mediated protein transport which is described in the present invention can be used for a broad range of applications. In particular, two basic approaches can be distinguished:

(i) Overexpression and/or enhancing the activity of SM proteins to increase the secretory transport capacity of a cell, or
(ii) reducing SM protein activity and/or expression as a means of gene therapy in order to reduce cancer cell proliferation and/or invasion.

### Applicability of SM Protein Overexpression:

The described invention describes a method to generate improved eukaryotic host cells for the production of heterologous proteins by improving the overall protein secretion capacity of cells by overexpression of proteins of the SM family.

This allows to increase protein yield in production processes based on eukaryotic cells. It thereby reduces the cost of goods of such processes and at the same time it reduces the number of batches that need to be produced to generate the material needed for research studies, diagnostics, clinical studies or market supply of a therapeutic protein. The invention furthermore speeds up drug development as often the generation of sufficient amounts of material for pre-clinical studies is a critical work package with regard to the timeline.

The invention can be used to increase the protein production capacity of all eukaryotic cells used for the generation of one or several specific proteins for either diagnostic purposes, research purposes (target identification, lead identification, lead optimization) or manufacturing of therapeutic proteins either on the market or in clinical development.

As shown in the present application, heterologous expression of SM proteins leads to increased production of all classes of proteins, including secreted enzymes, growth factors and antibodies. As transmembrane proteins share the same vesicle-mediated transport pathways which are regulated by the interplay of SM proteins and SNAREs, this engineering approach is equally applicable for improving the transport of transmembrane proteins and for enhancing their abundance on the cell surface.

Therefore, the method described herein can also be used for academic and industrial research purposes which aim to characterize the function of cell-surface receptors. E.g. it can be used for the production and subsequent purification, crystallization and/or analysis of surface proteins. Furthermore, transmembrane proteins generated by the described method or cells expressing these proteins can be used for screening assays, e.g. screening for substances, identification of ligands for orphan receptors or search for improved effectiveness during lead optimization. This is of crucial importance for the development of new human drug therapies as cell-surface receptors are a predominant class of drug targets.

Moreover, the method described herein can be advantageous for the study of intracellular signalling complexes associated with cell-surface receptors or the analysis of cell-cell-communication which is mediated in part by the interaction of soluble growth factors with their corresponding receptors on the same or another cell.

Applicability of Decreasing/Inhibiting SM Protein Expression and/or Activity:

In the present invention, we provide evidence that the reduction of SM expression leads to reduced secretion of soluble extracellular proteins, as shown for Munc18c and Sly1. This makes SM proteins attractive targets for therapeutic manipulation.

One of the hallmarks in the conversion from a normal healthy cell to a cancer cell is the acquisition of independency from the presence of exogenous growth factors. In contrast to the normal cell, tumor cells are able to produce all growth factors necessary for their survival and proliferation by themselves. In addition to this autocrine mechanism, cancer cells often show an upregulated expression of growth factor receptors on their surface, which leads to an increased responsiveness towards paracrine-acting growth and survival factors secreted from cells in the surrounding tissue. By targeting SM-proteins like Sly-1 and Munc18 in tumor cells, e.g. by using shRNA-, siRNA- or anti-sense RNA-approaches, it might be possible to disrupt autocrine as well as paracrine growth-stimulatory and/or survival mechanisms in two ways: (i) By reducing growth factor transport and secretion and (ii) by decreasing the amount of the corresponding growth factor-receptor on tumor cells. Thereby both, the amount of growth stimulating signal and the ability of the cancer cell to perceive and respond to these signals will be reduced. Inhibition of SM protein expression or activity in cancer cells should therefore represent a powerful tool to prevent cancer cell proliferation and survival.

SM proteins furthermore seem to be a potent therapeutic target to suppress tumor invasion and metastasis. During the later stages of most types of human cancer, primary tumors spawn pioneer cells that move out, invade adjacent tissues, and travel to distant sites where they may succeed in founding new colonies, known as metastasis.

As a prerequisite for tissue invasion, cancer cells express a whole set of proteases which enable them to migrate through the surrounding healthy tissue, to cross the basal membrane, to get into the blood stream and to finally invade the tissue of destination. Some of these proteases are expressed as membrane-bound proteins, e.g. MT-MMPs and ADAMs. Due to their crucial role in matrix remodelling, shedding of growth factors and tumor invasion, proteases themselves are discussed as drug targets for cancer therapy. We claim that inhibition of SM protein expression and/or activity in tumor cells reduces the amount of membrane-bound proteases on the surface of the targeted cell. This should decrease or even impair the invasive capacity of the tumor cell as well as its ability for growth factor shedding, resulting in reduced invasiveness and metastatic potential of the tumor. Thus, targeting proteins of the SM family offers a novel way of preventing late-stage tumorgenesis, especially the conversion from a benign/solid nodule to an aggressive, metastasizing tumor.

For therapeutic applications it is, thus, the goal to reduce and/or inhibit the activity and/or expression of SM proteins. This can be achieved either by a nucleotide composition which is used as human therapeutic to treat a disease by inhibiting the function of SM proteins whereby the drug is composed of an shRNA, RNAi, siRNA or an antisense RNA specifically inhibiting the SM protein through binding a sequence motive of its RNA. Reduction/inhibition of SM protein activity/expression can also be achieved by a drug substance containing nucleotides binding and silencing the promoter of the respective SM protein gene.

Furthermore, a drug substance or product can be composed of a new chemical entity or peptide or protein inhibiting expression or activity of a SM protein. In case of a protein being the active pharmaceutical compound it may be a (i) protein binding to the promoter of the SM protein thereby inhibiting its expression, (ii) protein binding to the SM protein or its interaction partner (e.g. a syntaxin or a protein within the SNARE complex) thereby hindering functional interactions of the SM protein with its binding partner, (iii) a protein similar to the SM protein which however does not fulfill its functions, meaning a “dominant-negative” SM protein variant, or (iv) a protein acting as scaffold for both the SM protein and its binding partner, resulting in irreversible binding of the proteins and the formation of a stable and unfunctional protein complex.

In accordance with the invention, there are provided novel methods of using the compounds of the present invention. Accordingly, the compounds of the present invention may be used to treat cancer or other abnormal proliferative diseases. Cancers are classified in two ways: by the type of tissue in which the cancer originates (histological type) and by primary site, or the location in the body where the cancer first developed. The most common sites in which cancer develops include the skin, lungs, female breasts, prostate, colon and rectum, the lymphoid system, cervix and uterus.

The compounds are thus useful in the treatment of a variety of cancers, including but not limited to the following:

AIDS-related cancer such as Kaposi's sarcoma; bone related cancer such as Ewing's family of tumors and osteosarcoma; brain related cancer such as adult brain tumor, childhood brain stem glioma, childhood cerebellar astrocytoma, childhood cerebral astrocytoma/malignant glioma, childhood ependymoma, childhood medulloblastoma, childhood supratentorial primitive neuroectodermal tumors, childhood visual pathway and hypothalamic glioma and other childhood brain tumors; breast cancer; digestive/gastrointestinal related cancer such as anal cancer, extrahepatic bile duct cancer, gastrointestinal carcinoid tumor, colon cancer, esophageal cancer, gallbladder cancer, adult primary liver cancer, childhood liver cancer, pancreatic cancer, rectal cancer, small intestine cancer and stomach (gastric) cancer; endocrine related cancer such as adrenocortical carcinoma, gastrointestinal carcinoid tumor, islet cell carcinoma (endocrine pancreas), parathyroid cancer, pheochromocytoma, pituitary tumor and thyroid cancer; eye related cancer such as intraocular melanoma, and retinoblastoma; genitourinary related cancer such as bladder cancer, kidney (renal cell) cancer, penile cancer, prostate cancer, transitional cell renal pelvis and ureter cancer, testicular cancer, urethral cancer, Wilms' tumor and other childhood kidney tumors; germ cell related cancer such as childhood extracranial germ cell tumor, extragonadal germ cell tumor, ovarian germ cell tumor and testicular cancer; gynecologic related cancer such as cervical cancer, endometrial cancer, gestational trophoblastic tumor, ovarian epithelial cancer, ovarian germ cell tumor, ovarian low malignant potential tumor, uterine sarcoma, vaginal cancer and vulvar cancer; head and neck related cancer such as hypopharyngeal cancer, laryngeal cancer, lip and oral cavity cancer, metastatic squamous neck cancer with occult primary, nasopharyngeal cancer, oropharyngeal cancer, paranasal sinus and nasal cavity cancer, parathyroid cancer and salivary gland cancer; hematologic/blood related cancer such as leukemias, such as adult acute lymphoblastic leukemia, childhood acute lymphoblastic leukemia, adult acute myeloid leukemia, childhood acute myeloid leukemia, chronic lymphocytic leukemia, chronic myelogenous leukemia and hairy cell leukemia; and lymphomas, such as AIDS-related lymphoma, cutaneous T-cell lymphoma, adult Hodgkin's lymphoma, childhood Hodgkin's lymphoma, Hodgkin's lymphoma during pregnancy, mycosis fungoides, adult non-Hodgkin's lymphoma, childhood non-Hodgkin's lymphoma, non-Hodgkin's lymphoma during pregnancy, primary central nervous system lymphoma, Sezary syndrome, cutaneous T-cell lymphoma and Waldenström's macroglobulinemia and other hematologic/blood related cancer such as chronic myeloproliferative disorders, multiple myeloma/plasma cell neoplasm, myelodysplastic syndromes and myelodysplastic/myeloproliferative diseases; lung related cancer such as non-small cell lung cancer and small cell lung cancer musculoskeletal related cancer such as Ewing's family of tumors, osteosarcoma, malignant fibrous histiocytoma of bone, childhood rhabdomyosarcoma, adult soft tissue sarcoma, childhood soft tissue sarcoma and uterine sarcoma; neurologic related cancer such as adult brain tumor, childhood brain tumor, brain stem glioma, cerebellar astrocytoma, cerebral astrocytoma/malignant glioma, ependmoma, medulloblastoma, supratentorial primitive neuroectodermal tumors, visual pathway and hypothalamic glioma and other brain tumors such as neuroblastoma, pituitary tumor and primary central nervous system lymphoma; respiratory/thoracic related cancer such as non-small cell lung cancer, small cell lung cancer, malignant mesothelioma, thymoma and thymic carcinoma; skin related cancer such as cutaneous T-cell lymphoma, Kaposi's sarcoma, melanoma, Merkel cell carcinoma and skin cancer.

These disorders have been well characterized in man, but also exist with a similar etiology in other mammals, and can be treated by pharmaceutical compositions of the present invention.

For therapeutic use, the compounds may be administered in a therapeutically effective amount in any conventional dosage form in any conventional manner. Routes of administration include, but are not limited to, intravenously, intramuscularly, subcutaneously, intrasynovially, by infusion, sublingually, transdermally, orally, topically or by inhalation, tablet, capsule, caplet, liquid, solution, suspension, emulsion, lozenges, syrup, reconstitutable powder, granule, suppository and transdermal patch. Methods for preparing such dosage forms are known (see, for example, H. C. Ansel and N. G. Popovish, Pharmaceutical Dosage Forms and Drug Delivery Systems, 5th ed., Lea and Febiger (1990)). A therapeutically effective amount can be determined by a skilled artisan based upon such factors as weight, metabolism, and severity of the affliction etc. Preferably the active compound is dosed at about 1 mg to about 500 mg per kilogram of body weight on a daily basis. More preferably the active compound is dosed at about 1 mg to about 100 mg per kilogram of body weight on a daily basis.

The compounds may be administered alone or in combination with adjuvants that enhance the stability of the inhibitors, facilitate administration of pharmaceutic compositions containing them in certain embodiments, provide increased dissolution or dispersion, increase inhibitory activity, provide adjunct therapy, and the like. Advantageously, such combinations may utilize lower dosages of the active ingredient, thus reducing possible toxicity and adverse side effects.

Pharmaceutically acceptable carriers and adjuvants for use with compounds according to the present invention include, for example, ion exchangers, alumina, aluminum stearate, lecithin, serum proteins, buffer substances, water, salts or electrolytes and cellulose-based substances. This is not a complete list possible pharmaceutically acceptable carriers and adjuvants, and one of ordinary skilled in the art would know other possibilities, which are replete in the art.

In summary, the present invention describes a novel method for enhancing the secretory transport of proteins in eukaryotic cells by heterologous expression of Munc18c, Sly1 or other members of the SM protein family and combinations thereof. This method is particularly useful for the generation of optimized host cell systems with enhanced production capacity for the expression and manufacture of recombinant protein products.

Sec1/Munc18 (SM) proteins are required for membrane fusion in intracellular protein transport, but the nature of their action has long been proposed as diverse rather than united, in part because of the heterogeneity of the interactions between SM proteins and SNAREs. In this invention we assess the physiological impact of two SM proteins on the secretory pathway. A fundamental finding is that Munc18c and Sly1, involved in vesicle fusion to the plasma membrane and the Golgi, unanimously stimulate overall exocytosis.

Consistent with this model, we show that overall exocytosis is reduced when Sly1 and Munc18c are knocked down (FIG. 3). In contrast, elevated levels of Sly1 by overexpression increase the secretion capacity (FIG. 4). Importantly and surprisingly, Munc18c significantly stimulate secretion capacity of host cells as well. In support of this, we demonstrated that Munc 18c directly binds to SNARE complexes specialized for fusion to the PM (plasma membrane) (FIG. 5).

Previous studies assigned an inhibitory role for Munc18c in exocytosis (Riento et al., 2000; Kanda et al., 2005; Tellam et al., 1997; Thurmond et al., 1998), which is contradicted by the results of the present invention. To provide molecular insight into Munc18c's role in the trafficking machinery, in particular its interaction with exocytic SNARE proteins consisting of syntaxin 4, SNAP-23 and VAMP2, we report immunoprecipitation experiments. As shown in FIG. 5, Munc18c-specific antibodies quantitatively precipitate the Munc18c along with a significant fraction of syntaxin4, SNAP-23 and VAMP 2, indicating the in vivo association of Munc18c with these SNAREs, which facilitate vesicle-organelle fusion in the secretory pathway (Peng and Gallwitz, 2002; Shen et al., 2007; Scott et al., 2004). This finding highlights that, similar to Sly1, which binds to the fully assembled SNARE complexes and facilitates fusion the Golgi apparatus, Munc18c directly interacts with SNARE complexes as well, suggesting a conserved mechanism of action by promoting the SNARE-mediated trafficking machinery.

So, both the physiological role and the mechanism of Sly1 and Munc18c function are conserved in SNARE-mediated secretory pathway.

SM protein-based secretion engineering enhances exocytosis of a variety of proteins including enzymes, growth hormones and immunotherapeutic monoclonal antibodies when Sly1, Munc18c and the general organelle-expanding factor Xbp-1 are overexpressed.

The data of the present application demonstrate an additive or even synergistic effect on protein secretion upon simultaneous overexpression of two SM proteins within the same cell, as shown for Munc18c and Sly1. Our data thus support a model for united functions of SM proteins in stimulating SNARE-mediated trafficking machinery and represents a novel strategy of posttranslational engineering for enhanced secretion.

Taken together, in the present application we provide first and surprising evidence for a united, activation role of SM proteins in the exocytic/secretory pathway. Based on this finding, we pioneer an SM protein-based posttranslational engineering by which enhanced exocytosis is successfully achieved.

Efficient production of protein therapeutics remains a big challenge to biotechnology industry. So far, a variety of different metabolic engineering strategies have been developed. For instance, by increasing transcription (transcription engineering); by modulating translation performance of mammalian cells (translation engineering); by boosting production of specific glycoforms (glycosylation engineering); by exclusively redirecting metabolic energy to product formation (controlled proliferation technology) and by improving the viability of production cell lines (anti-apoptosis engineering). However, metabolic engineering based on orchestrated secretion machinery has remained elusive. Based on the finding that Sly1 and Munc18c unanimously stimulate overall exocytosis, we report here for the first time an SM protein-based posttranslational engineering that leads to enhanced secretory capacity of mammalian cells. The system is independent of the expression configuration, the type of promoter used and the promoter-mediated transcription level, making it especially suitable for the industrial production of recombinant proteins and pharmaceuticals.

The present invention furthermore provides a means to inhibit or reduce protein exocytosis by interfering with SM protein expression. This should provide useful means for the treatment of cancer or inflammatory conditions.

Previously, it has been described that in eukaryotic cells, membrane-bound transport vesicles shuttle proteins and lipids between subcellular compartments/organelles. At each trafficking step, SNARE [soluble NSF (N-ethylmaleimide sensitive factor) attachment receptor] proteins from both vesicles and target membranes form trans-SNARE complexes that constitute the core machinery required for fusion to occur. To meet the physiological requirements in various situations, the SNARE-mediated fusion machinery must be spatially and temporally tunable in order for stimuli from both intracellular and extracellular sources to be integrated properly. Thus, it is crucial that the function of SNARE is modulated or fine-tuned in vivo so that neither the specificity nor the speed of membrane fusion are compromised. Sec1/Munc18 (SM) proteins could hold the key to regulating SNARE proteins. First identified in yeast and nematodes, SM proteins are essential for fusion. The fact that there are few interaction partners other than SNAREs has led to the prevalent notion that SM proteins are functionally coupled to SNARE proteins (Gallwitz and Jahn, 2003; Jahn et al., 2003; Toonen and Verhage, 2003). However, the attempt to generalize a functional model for SM proteins has been considerably hampered by the heterogeneous nature of their interactions with SNAREs. At distinct trafficking steps and in different organisms, monomer syntaxins (Dulubova et al., 1999; Yang et al., 2000; Peng and Gallwitz, 2002), vesicle-associated SNARE (Li et al., 2005; Carpp et al., 2006; Peng and Gallwitz; 2004; Shen et al., 2007), the heterodimer t-SNARE complexes (Scott et al., 2004; Zilly et al., 2006) as well as ternary, fully assembled SNARE complexes (Carpp et al., 2006; Peng and Gallwitz; 2004; Shen et al., 2007; Togneri et al., 2006; Carr et al., 1999; Dulubova et al., 2007) have been shown to bind easily to an individual SM protein. As a consequence, the physiological significance of these interactions has been interpreted both positively and negatively for SM protein function in membrane fusion.

Consequently, the molecular mechanism, especially the physiological role of the SM proteins in the secretory pathway, is still enigmatic. For example, Sly1 interacts with monomer syntaxin 5, monomer vesicle-bound SNAREs and fully assembled SNARE complexes (Li et al., 2005; Peng and Gallwitz; 2004), and has been shown to positively influence the formation of SNARE complexes and fusion specificity (Peng and Gallwitz, 2002; Kosodo et al., 2002).

On the other hand, previous studies assigned an inhibitory role for Munc18 proteins in membrane fusion and exocytosis: The neuron-specific Munc18a, which is specifically required for regulated exocytosis of synaptic vesicles, assumes two functionally contradictory interactions with SNAREs: by binding to the closed conformation of syntaxin 1, thus inhibiting SNARE complex assembly (Dulubova et al., 1999; Yang et al., 2000), and to fully assembled SNARE complexes, therefore promoting membrane fusion (Shen et al., 2007; Dulubova et al., 2007). Consistently, both inhibitory and promotive effects of Munc18a on exocytosis were reported (Wu et al., 1998; Verhage et al., 2000; Voets et al., 2001). Munc18b and Munc18c are homologous in sequences to Munc18a but expressed ubiquitously. In vitro data indicated Munc 18c is similar to Munc 18a in SNARE binding (Latham et al., 2006; D'Andrea-Merrins et al., 2007), and the structures of the two proteins are conserved (Misura et al., 2000; Hu et al., 2007). Genetic and physiological studies, however, have so far provided exclusive evidence for an inhibitory role in exocytosis by Munc18b and Munc18c (Riento et al., 2000; Kanda et al., 2005; Tellam et al., 1997; Thurmond et al., 1998). For instance, 1) overexpression of Munc18a in flies inhibits neuron transmission (Wu et al., 1998), 2) overexpression of Munc18b in Caco-2 cells inhibits the apical delivery of influenza virus hemagglutinin (Riento et al., 2000), 3) Munc18c competes the binding to syntaxin 4 with VAMP2 (Thurmond et al., 1998); 4) translocation of insulin-stimulated GLUT vesicles in adipocytes was inhibited by overexpression of Munc18c but enhanced in Munc18c-null mice (Tellam et al., 1997; Thurmond et al., 1998).

In contrast to these reports and in discrepancy to the ruling preconception, in the present application we demonstrate a novel and surprising role for the two SM proteins Sly1 and Munc 18c by demonstrating that both proteins equally stimulate exocytosis in general. The molecular mechanism of the activation role by Munc18c and Sly1 is likely conserved too. Based on these surprising findings, we pioneered an SM protein-based secretion engineering that results in enhanced secretion in mammalian cells. The SM protein-based secretion engineering represents a novel strategy of metabolic engineering and provides a new platform for the manufacturing of protein pharmaceuticals in industry.

In particular, the positive effect of Sly1 and Munc18c expression on the secretory capacity of mammalian cells points to a novel, post-translational approach to engineer mammalian production cell lines for increased secretion.

It is demonstrated in example 5 that simultaneous overexpression of sly1 and munc18c leads to an 8-fold increase in SEAP production, as compared to the 5-fold by sly1 or munc18c alone (FIG. 4a,). Secretion of SAMY and VEGF₁₂₁ is also increased (FIGS. 4b, 4c). Overexpression of sly1, munc18c and xbp-1 altogether increases secretion of SEAP, SAMY and VEGF by 10-, 12- and 8-fold, respectively (FIGS. 4a, 4b, 4c), clearly demonstrating the existence of a synergistic effect on secretion between Sly1 and Munc18c, and between the two SM proteins and the general organelle-expanding factor Xbp-1.

It is further demonstrated in example 6 that by generation of stable CHO-K1-derived cell lines engineered for constitutive expression of either sly1 (CHO-Sly1₁₆ and CHO-Sly1₂₃) or munc18c (CHO-Munc18c₈ and CHO-Munc18c₉), CHO-Sly1₁₆ and CHO-Sly1₂₃ stimulate SEAP secretion by a factor of 4- and 8-fold (FIG. 6a) and SAMY production 4- and 5-fold (FIG. 6b). Interestingly, CHO-Sly1₂₃ producing more SEAP also shows higher Sly1 levels suggesting a positive correlation of SM and product proteins (FIG. 6c). Similarly, cells transgenic for constitutive munc18c expression (CHO-Munc18c₉) produce 9- and 6.5-fold more SEAP and SAMY (FIGS. 6e and 6f) and CHO-Munc18₉ producing more SEAP also shows higher Munc18c levels (FIG. 6d). The stable cell lines CHO-Sly1-Munc18c₁, double-transgenic for constitutive Sly1 and Munc18c expression and CHO-Sly1-Munc18c-Xbp-1₇, triple-transgenic for constitutive Sly1, Munc18c and Xbp-1 expression show 13- and 16-fold higher SEAP production compared to parental CHO-K1 (FIG. 6g).

Particularly, SM protein-based secretion engineering increases specific antibody productivity of production cell lines. Example 7 illustrates this by using SM protein-based secretion engineering in a prototype biopharmaceutical manufacturing scenario to express monoclonal anti-human CD20 IgG1 known as Rituximab in CHO-Sly1₁₆ and CHO-Sly1₂₃ (up to 10-fold increase), in CHO-Sly1-Munc18c₁ (up to 15-fold increase) and in CHO-Sly1-Xbp-1₄ (up to 13-fold increase) and in CHO-Sly1-Munc18c-Xbp-1₇ (up to 19-fold increase) (FIG. 7a). When producing Rituximab in CHO-Sly1-Munc18c-Xbp-1₇ ad hoc production levels of up to 40 pg/cell/day can be reached, which corresponds to a near 20-fold increase compared to an isogenic control cell line (FIG. 7a). SDS-PAGE analysis indicate that the antibodies produced by CHO-Sly1-Munc18c-Xbp-1₇ and wild-type CHO-K1 cells are structurally intact and indistinguishable from each other (FIGS. 7b, 7c). Maldi-TOF-based Glycoprofiling of N-linked Fc oligosaccharides from Rituximab produced in CHO-Sly1-Munc18c-Xbp-1₇ reveals no difference compared to native production cell lines indicating that SM/Xbp-1-based secretion engineering is not compromising the product quality (FIGS. 7d and 7e).

### DESCRIPTION OF THE FIGURES

FIG. 1

Expression and localization of Sly1 and Munc18 in HEK-293. (a) and (b) RT-PCR-based detection of sly1 (a) and munc18 (b) transcripts using actin as an endogenous control. 1-Kb ladder is used as size standard. (c) Western blot of Munc 18a/b/c. (d) Confocal micrographs showing the subcellular localization of Sly1 and Munc18c in HEK-293 transfected with sets of YFP-Munc18c (pRP23) and CFP-Syntaxin 4 (Stx4, pRP29) or YFP-Sly1 (pRP32) and CFP-Syntaxin 5 (Stx5, pRP40). The arrows indicate either colocalization of Sly1 and syntaxin 5 (upper panel) or Munc 18c and syntaxin 4 (lower panel).

FIG. 2

shRNA-based knockdown of sly1 and Munc18c. (a) Schematic diagram of the dicistronic sly1-/GFP-encoding expression vector pRP3 used as sly1-specific knockdown reporter construct for different sly1-specific shRNAs. (b) Fluorescence micrographs of CHO-K1 co-transfected with pRP3 and different shRNA-encoding expression vectors and cultivated for 48 h. (c) Schematic diagram of the dicistronic Munc18c-/GFP-encoding expression vector pRP4 used as Munc 18c-specific knockdown reporter construct for different Munc18c-specific shRNAs. Fluorescence micrographs of HEK-293 co-transfected with pRP4 and different shRNA-encoding expression vectors and cultivated for 48 h.

FIG. 3

shRNA-based knockdown of sly1 and munc18c decreases overall exocytosis. (a) Sly1-specific Western blot of HEK-293 transfected with sly1-targeted shRNA expression vectors (shRNA_{sly1}_{_{—}}_{1/2/3}; pRP5-7). The parental vector pmU6, control shRNA and p27^{Kip1} are used as control. (b) SEAP expression profile of HEK-293 co-transfected with pSEAP2-Control and different shRNA_{sly1}, expression vectors (48 h). (c) Munc18c-specific Western blot of HEK-293 transfected with munc18c-targeted shRNA expression vectors (shRNA_{munc18c}_{_{—}}_{1/2/3}; pRP12, 14, 38, 39). (d) SEAP expression profile of HEK-293 co-transfected with pSEAP2-Control and different shRNA_{munc18} expression vectors.

FIG. 4

Ectopic expression of Sly1 and Munc18c post-transcriptionally boosts protein production of CHO-K1. (a-c) Production profiles of CHO-K1 co-transfected with SEAP (pSEAP1-Control) (a), SAMY (pSS158) (b) or VEGF₁₂₁ (pWW276) (c) production vectors and (different combinations of) Sly1-(pRP24), Munc18c-(pRP17) and Xbp-1 (pcDNA3.1-Xbp-1)-encoding expression vectors. (d) Quantitative RT-PCR-based profiling of product mRNA levels in the presence or absence of SM protein expression.

FIG. 5

Interaction of Munc 18c with exocytic SNARE complexes. Western blot analysis of Munc18c, syntaxin4, SNAP-23 and VAMP2/synaptobrevin 2 (SybII) following immunoprecipitation of HEK-293 lysates using affinity-purified, protein A-sepharose-coupled anti-Munc18c antibodies. Non-precipitated protein (supernatant) as well as Sly1 is used as control.

FIG. 6

SM protein-based secretion engineering enhances production of heterologous proteins in CHO-K1-derived cell lines. (a) SEAP production of stable mixed and clonal CHO-K1-derived populations transgenic for constitutive Sly1 and SEAP expression (CHO-Sly1₁₆ and CHO-Sly1₂₃ and CHO-Sly1ₘᵢₓ) cultivated for 48 h. (b) SAMY production of CHO-Sly1₁₆ and CHO-Sly1₂₃ and CHO-Sly1ₘᵢₓ transiently transfected with pSS158. (c) Sly1-specific Western blot of CHO-K1, CHO-Sly1₁₆ and CHO-Sly1₂₃ with p27^{Kip1} as loading control. (d) Munc18c-specific Western blot of CHO-K1, CHO-Munc18c₈ and CHO-Munc 18c₉ with p27^{Kip1} as loading control. (e) SEAP production of stable mixed and clonal CHO-K1-derived populations transgenic for constitutive Munc18c and SEAP expression (CHO-Munc 18c₈, CHO-Munc 18c₉ and CHO-Munc 18cₘᵢₓ) cultivated for 48 h. (f) SAMY production CHO-Munc 18c₈, CHO-Munc 18c₉ and CHO-Munc 18ₘᵢₓ transiently transfected with pSS158. (g) SEAP production profiles of stable cell clones constitutively expressing Sly1 and Munc18c (CHO-Sly1-Munc18c₁), Sly1 and Xbp-1 (CHO-Sly1-Xbp1₄) and Sly1, Munc18c as well as Xbp-1 (CHO-Sly1-Munc18c-Xbp-1₇) cultivated for 48 h.

FIG. 7

Production and glycoprofiling of Rituximab produced in secretion-engineered CHO-K1 derivatives. (a) Specific Rituximab productivity of different secretion-engineered CHO-K1 derivatives. (Increased secretion of human IgG1 by SM protein-based metabolic engineering. (b, c) Rituximab purified from CHO-Sly1-Munc18c-Xbp-1₇ and CHO-K1 cells are analyzed by non-reducing (b) and reducing (c) SDS-PAGE. The molecular weight (KDa) of standard proteins and the heavy and light chains (HC, LC) of the IgG1 are shown. (d, e) MALDI-TOF-based glycoprofiling of Rituximab produced in CHO-K1 and secretion-engineered CHO-Sly1-Munc 18c-Xbp-1₇.

FIG. 8

Schematic drawing of expression constructs:

Vector encoding at least one protein of interest (GOI) and one SM protein from separate expression units (a) or from one bi-cistronic unit (b).

Expression vector comprising genes of two SM proteins encoded either from separate expression cassettes (c) or bi-cistronically, whereby the two genes are linked via an IRES element (d).

Expression vector encoding at least two SM proteins and a gene of interest (e) or several SM proteins from one multi-cistronic expression unit.

FIG. 9

SM proteins enhance HRP secretion from human cells:

Measurement of HRP activity in supernatants of human HT1080 cells co-transfected with secreted horseraddish peroxidase (ssHRP) and empty vector (Mock, black bars), Munc18c (grey bars), Sly1 (shaded bars) or a bi-cistronic construct encoding Munc18c and Sly1 (Munc-IRES-Sly, striped bars). Relative ssHRP titers measured at 24 and 48 h post-transfection as well as specific productivities are plottet relative to the Mock control which was set 1.0. The values correspond to the mean of triplicate samples, error bars=SEM.

FIG. 10

Overexpression of SM proteins in IgG producer cell lines increases specific productivities and final IgG titers

(A) Relative specific IgG1 productivities of cells stably expressing either an empty vector (Mock) or expression constructs for Sly-1 (Sly1), Munc-18c (Munc) or both SM proteins (Munc/Sly1). The productivities were calculated from titers and viable cell counts during a fed-batch production process. The bars represent mean values of n=2 (Mock) to n=6 monoclonal transgenic IgG production cell lines and are depicted relative to the specific productivities in Mock cells which were set 100%.

(B) IgG titers from stable cell populations stably expressing the described constructs over a 9 day fed-batch fermentation process.

### DETAILED DESCRIPTION OF THE INVENTION

The general embodiments “comprising” or “comprised” encompass the more specific embodiment “consisting of”. Furthermore, singular and plural forms are not used in a limiting way.

Terms used in the course of this present invention have the following meaning.

The term “gene” means a desoyribonucleic acid (DNA) sequence (e.g. cDNA, genomic DNA or mRNA). In the present invention, gene refers preferredly to a human DNA sequences, but included are equally homologous sequences from other mammalian species, preferredly mouse, hamster and rat, as well as homologous sequences from additional eucaryotic species including chicken, duck, moss, worm, fly and yeast.

The collective term “Sec1/Munc-18 proteins” or “SM proteins” or Sec1/Munc18 group of proteins” or SM-proteins or “genes encoding SM-proteins” or “SM family” comprises a family of hydrophilic proteins of 60-70 kDa which share a high degree of structural similarity and are evolutionary conserved from yeast to men.

Munc18 and Sly1 both belong to the family of Sec1/Munc18 proteins. This family further includes up to now:

in yeast: Sec1p, Sly1 p, Vps33p and Vps45p
in drosophila: ROP, Sly1 and Vps33/carnation
in nematodes: Unc-18 as well as 5 other genes according to genome sequence databases
in vertebrates: Munc18-1, -2 and -3, VPS45, VPS33-A and -B and Sly1.

The term SM-proteins also encompasses derivatives, mutants and fragments of such proteins, e.g. a flag-tagged, HIS-tagged or otherwise tagged SM-protein. Such derivatives are frequently used, e.g. to ease purification or isolation or visualization of the protein.

SM proteins show a high homology over the entire sequence, suggesting that they might exhibit similar overall structures. Furthermore, loss-of-function mutations have been described for nine SM genes in four species, which all lead to severe impairment of vesicle trafficking and fusion, indicating that SM proteins play similar and central roles in the process of vesicle transport and secretion.

The examples of the present invention use Munc18 and Sly1 as model proteins, however, the present invention can be equally well transferred to other members of the SM protein family.

Furthermore, in light of the high degree of conservation across species, SM proteins can be used to modulate secretion and cell-surface expression of proteins in all eukaryotic host cell species from yeast over worms and insect cells to mammalian systems.

In eukaryotic cells, membrane-bound transport vesicles shuttle proteins and lipids between subcellular compartments/organelles. The fusion of cellular transport vesicles with the cell membrane or with a target compartment (such as a lysosome, the Golgi complex or the plasma membrane) is mediated by SNARE [soluble NSF (N-ethylmaleimide sensitive factor) attachment receptor] proteins. To meet the physiological requirements of the cell and to maintain the compartment-specific membrane composition, the SNARE-mediated fusion machinery is spatially and temporally controlled by small proteins of the Sec1/Munc18 (SM) family. By direct binding to SNAREs and Syntaxins, SM proteins regulate all steps of vesicle-mediated transport between intracellular compartments/organelles and the plasma membrane.

The term “Munc-18” or “Munc-18 protein(s)” or “Munc-18 protein family” includes all Munc-18 genes and gene products/proteins present in eucaryotic organisms. This explicitly includes the three Munc-18 paralogs, namely Munc-18a (which is also called “Munc-18-1”), Munc-18b and Munc-18c, which have evolved in vertebrates.

More specifically, the term “Munc-18c” refers to the human gene and protein Munc18c which is also known as “Syntaxin binding protein 3” (STXBP3) or “Platelet Sec1 Protein” (PSP), SEQ-ID NO 39, including its homologs in other mammalian species, including mouse, hamster, rat, dog and rabbit.

The term “Sly-1” or “Sly-1 protein(s)” refers to all Sly1 genes and proteins expressed from these genes in vertebrates, preferredly mammals. More specifically “Sly-1” refers to the human Sly1 protein, also known as “Sec1 family domain containing protein 1” (SCFD1) or “Syntaxin binding protein-1 like protein 2” (STXBP1L2), SEQ-ID NO. 41

The term “XBP-1” equally refers to the XBP-1 DNA sequence and all proteins expressed from this gene, including XBP-1 splice variants. Preferentially, XBP-1 refers to the human XBP-1 sequence and preferredly to the spliced and active form of XBP-1, also called “XBP-1(s)”. The transcription factor XBP-1 is known to be one of the key-regulators of secretory cell differentiation as well as maintenance of ER homeostasis and expansion (Lee, 2005; Iwakoshi, 2003). These functions make XBP-1 a candidate for secretion engineering approaches.

More specifically “XBP-1” refers to the human XBP-1 protein, SEQ-ID NO. 43.

The term “productivity” or “specific productivity” describes the quantity of a specific protein which is produced by a defined number of cells within a defined time. The specific productivity is therefore a quantitative measure for the capacity of cells to express/synthesize/produce a protein of interest. In the context of industrial manufacturing, the specific productivity is usually expressed as amount of protein in picogram produced per cell and day (‘pg/cell*day’ or ‘pcd’).

One method to determine the “specific productivity” of a secreted protein is to quantitatively measure the amount of protein of interest secreted into the culture medium by enzyme linked immunosorbent assay (ELISA). For this purpose, cells are seeded into fresh culture medium at defined densities. After a defined time, e.g. after 24, 48 or 72 hours, a sample of the cell culture fluid is taken and subjected to ELISA measurement to determine the titer of the protein of interest. The specific productivity can be determined by dividing the titer by the average cell number and the time.

Another example how to measure the “specific productivity” of cells is provided by the homogenous time resolved fluorescence (HTRF®) assay.

“Producitvity” of cells for an intracellular, membrane-associated or transmembrane protein can also be detected and quantified by Western Blotting. The cells are first washed and subsequently lysed in a buffer containing either detergents such as Triton-X, NP-40 or SDS or high salt concentrations. The proteins within the cell lysate are than separated by size on SDS-PAGE, transferred to a nylon membrane where the protein of interest is subsequently detected and visualized by using specific antibodies.

Another method to determine the “specific productivity” of a cell is to immunologically detect the protein of interest by fluorescently labeled antibodies raised against the protein of interest and to quantify the fluorescence signal in a flow cytometer. In case of an intracellular protein, the cells are first fixed, e.g. in paraformaldehyde buffer, and than permeabilized to allow penetration of the detection antibody into the cell. Cell surface proteins can be quantified on the living cell without need for prior fixation or permeabilization.

The “productivity” of a cell can furthermore by determined indirectly by measuring the expression of a reporter protein such as the green fluorescent protein (GFP) which is expressed either as a fusion protein with the protein of interest or from the same mRNA as the protein of interest as part of a bi-, tri-, or multiple expression unit.

The term “enhancement/increase of productivity” comprises methods to increase/enhance the specific productivity of cells. The specific productivity is increased or enhanced, if the productivity is higher in the cells under investigation compared to the respective control cells and if this difference is statistically significant. The cells under investigation can be heterogenous populations or clonal cell lines of treated, transfected or genetically modified cells; untreated, untransfected or unmodified cells can serve as control cells. In the context of a secreted protein of interest, the terms “enhanced/increased/improved productivity” and “enhanced/increased/improved exocytosis” and “enhanced/increase/improved secretion” have the same meaning and are used interchangeably.

The term “derivative” in general includes sequences suitable for realizing the intended use of the present invention, which means that the sequences mediate the increase in secretory transport in a cell.

The term “derivative” as used in the present invention means a polypeptide molecule or a nucleic acid molecule which is at least 70% identical in sequence with the original sequence or its complementary sequence. Preferably, the polypeptide molecule or nucleic acid molecule is at least 80% identical in sequence with the original sequence or its complementary sequence. More preferably, the polypeptide molecule or nucleic acid molecule is at least 90% identical in sequence with the original sequence or its complementary sequence. Most preferred is a polypeptide molecule or a nucleic acid molecule which is at least 95% identical in sequence with the original sequence or its complementary sequence and displays the same or a similar effect on secretion as the original sequence.

Sequence differences may be based on differences in homologous sequences from different organisms. They might also be based on targeted modification of sequences by substitution, insertion or deletion of one or more nucleotides or amino acids, preferably 1, 2, 3, 4, 5, 7, 8, 9 or 10. Deletion, insertion or substitution mutants may be generated using site specific mutagenesis and/or PCR-based mutagenesis techniques. The sequence identity of a reference sequence can be determined by using for example standard “alignment” algorithms, e.g. “BLAST”. Sequences are aligned when they fit together in their sequence and are identifiable with the help of standard “alignment” algorithms.

Furthermore, in the present invention the term “derivative” means a nucleic acid molecule (single or double strand) which hybridizes to other nucleic acid sequences. Preferably the hybridization is performed under stringent hybridization- and washing conditions (e.g. hybridisation at 65° C. in a buffer containing 5×SSC; washing at 42° C. using 0.2×SSC/0.1% SDS).

The term “derivatives” further means protein deletion and/or insertion mutants, phosphorylation mutants especially at a serine, threonine or tyrosine position and mutants bearing deletions of a binding site for protein kinase C (PKC) or casein kinase II (CKII).

The term “activity” describes and quantifies the biological functions of the protein within the cell or in in vitro assays

One assay for measuring the “activity” of an SM protein is a secretion assay e.g. for a model protein, an antibody or a protein of interest. Cells are cotransfected with ss-HRP-Flag plasmid together with either an empty vector or a gene under investigation such us Munc-18c or Sly-1. 24 h post-transfection cells are washed with serum-free media and HRP secretion is quantified after 0, 1, 3 and 6 h by incubation of clarified cell supernatant with ECL reagent. Measurements are done with a luminometer (Lucy2, Anthos) at 450 nm.

Another method for detection of the “activity” in terms of functional binding of an SM protein is to show the binding of an SM protein to its known interaction partner e.g. the binding of Munc-18c to Syntaxin-4 or physical interaction of Sly1 with Syntaxin-5. Binding of SM proteins to other proteins can be demonstrated by co-immunoprecipitation, e.g. pull-down of the SM protein using specific antibodies coupled to beads, denaturation of the beads and following separation and detection of co-immunoprecipitating proteins by SDS-PAGE and Western Blot.

Direct binding of SM proteins to another protein, e.g. syntaxins, can further be detected in yeast-two-hybrid assays. In this assay, both proteins are expressed in yeast cells as fusion proteins with DNA-binding and transactivation domain, respectively, of one transcription factor. Direct interaction of both proteins leads to a reconstitution of the transcription factor whose activity is detected colourimetrically or by the ability of the yeast cell to grow under selective conditions.

Another, yet indirect, method is provided by co-immunofluorescence of SM proteins and its binding partners and detection of their co-localization within the cell.

One method to measure the “activity” of XBP-1 is to perform band-shift experiments to detect binding of the XBP-1 transcription factor to its DNA binding site. Another method is to detect translocation of the active XBP-1 splice variant from the cytosol to the nucleus. Alternatively, XBP-1 “activity” can be indirectly confirmed by measuring induced expression of a bona fide XBP-1 target gene such as binding protein (BiP) upon heterologous expression of XBP-1.

“Host cells” in the meaning of the present invention are cells such as hamster cells, preferably BHK21, BHK TK⁻, CHO, CHO Pro-5, the CHO derived mutant cell lines Lec1 to Lec35, CHO-K1, CHO-DUKX, CHO-DUKX B1, and CHO-DG44 cells or the derivatives/progenies of any of such cell line. Particularly preferred are CHO-DG44, CHO-DUKX, CHO-K1 and BHK21, and even more preferred CHO-DG44 and CHO-DUKX cells. In a further embodiment of the present invention host cells also mean murine myeloma cells, preferably NSO and Sp2/0 cells or the derivatives/progenies of any of such cell line. Examples of murine and hamster cells which can be used in the meaning of this invention are also summarized in Table 1. However, derivatives/progenies of those cells, other mammalian cells, including but not limited to human, mice, rat, monkey, and rodent cell lines, or eukaryotic cells, including but not limited to yeast, insect, plant and avian cells, can also be used in the meaning of this invention, particularly for the production of biopharmaceutical proteins.

TABLE 1
Eukaryotic production cell lines

| CELL LINE | ORDER NUMBER |
|---|---|
| NS0 | ECACC No. 85110503 |
| Sp2/0-Ag14 | ATCC CRL-1581 |
| BHK21 | ATCC CCL-10 |
| BHK TK⁻ | ECACC No. 85011423 |
| HaK | ATCC CCL-15 |
| 2254-62.2 (BHK-21 derivative) | ATCC CRL-8544 |
| CHO | ECACC No. 8505302 |
| CHO wild type | ECACC 00102307 |
| CHO-K1 | ATCC CCL-61 |
| CHO-DUKX | ATCC CRL-9096 |
| (═CHO duk⁻, CHO/dhfr⁻) | |
| CHO-DUKX B11 | ATCC CRL-9010 |
| CHO-DG44 | (Urlaub et al., 1983) |
| CHO Pro-5 | ATCC CRL-1781 |
| Lec13 | (Stanley P. et al, 1984). |
| V79 | ATCC CCC-93 |
| B14AF28-G3 | ATCC CCL-14 |
| HEK 293 | ATCC CRL-1573 |
| COS-7 | ATCC CRL-1651 |
| U266 | ATCC TIB-196 |
| HuNS1 | ATCC CRL-8644 |
| Per.C6 | (Fallaux, F. J. et al, 1998) |
| CHL | ECACC No. 87111906 |

Host cells are most preferred, when being established, adapted, and completely cultivated under serum free conditions, and optionally in media which are free of any protein/peptide of animal origin. Commercially available media such as Ham's F12 (Sigma, Deisenhofen, Germany), RPMI-1640 (Sigma), Dulbecco's Modified Eagle's Medium (DMEM; Sigma), Minimal Essential Medium (MEM; Sigma), Iscove's Modified Dulbecco's Medium (IMDM; Sigma), CD-CHO (Invitrogen, Carlsbad, Calif.), CHO—S-Invtirogen), serum-free CHO Medium (Sigma), and protein-free CHO Medium (Sigma) are exemplary appropriate nutrient solutions. Any of the media may be supplemented as necessary with a variety of compounds examples of which are hormones and/or other growth factors (such as insulin, transferrin, epidermal growth factor, insulin like growth factor), salts (such as sodium chloride, calcium, magnesium, phosphate), buffers (such as HEPES), nucleosides (such as adenosine, thymidine), glutamine, glucose or other equivalent energy sources, antibiotics, trace elements. Any other necessary supplements may also be included at appropriate concentrations that would be known to those skilled in the art. In the present invention the use of serum-free medium is preferred, but media supplemented with a suitable amount of serum can also be used for the cultivation of host cells. For the growth and selection of genetically modified cells expressing the selectable gene a suitable selection agent is added to the culture medium.

The term “protein” is used interchangeably with amino acid residue sequences or polypeptide and refers to polymers of amino acids of any length. These terms also include proteins that are post-translationally modified through reactions that include, but are not limited to, glycosylation, acetylation, phosphorylation or protein processing. Modifications and changes, for example fusions to other proteins, amino acid sequence substitutions, deletions or insertions, can be made in the structure of a polypeptide while the molecule maintains its biological functional activity. For example certain amino acid sequence substitutions can be made in a polypeptide or its underlying nucleic acid coding sequence and a protein can be obtained with like properties.

The term “polypeptide” means a sequence with more than 10 amino acids and the term “peptide” means sequences up to 10 amino acids length.

The present invention is suitable to generate host cells for the production of biopharmaceutical polypeptides/proteins. The invention is particularly suitable for the high-yield expression of a large number of different genes of interest by cells showing an enhanced cell productivity.

“Gene of interest” (GOI), “selected sequence”, or “product gene” have the same meaning herein and refer to a polynucleotide sequence of any length that encodes a product of interest or “protein of interest”, also mentioned by the term “desired product”. The selected sequence can be full length or a truncated gene, a fusion or tagged gene, and can be a cDNA, a genomic DNA, or a DNA fragment, preferably, a cDNA. It can be the native sequence, i.e. naturally occurring form(s), or can be mutated or otherwise modified as desired. These modifications include codon optimizations to optimize codon usage in the selected host cell, humanization or tagging. The selected sequence can encode a secreted, cytoplasmic, nuclear, membrane bound or cell surface polypeptide.

The “protein of interest” includes proteins, polypeptides, fragments thereof, peptides, all of which can be expressed in the selected host cell. Desired proteins can be for example antibodies, enzymes, cytokines, lymphokines, adhesion molecules, receptors and derivatives or fragments thereof, and any other polypeptides that can serve as agonists or antagonists and/or have therapeutic or diagnostic use. Examples for a desired protein/polypeptide are also given below.

In the case of more complex molecules such as monoclonal antibodies the GOI encodes one or both of the two antibody chains.

The “product of interest” may also be an antisense RNA, siRNA, RNAi or shRNA.

“Proteins of interest” or “desired proteins” are those mentioned above. Especially, desired proteins/polypeptides or proteins of interest are for example, but not limited to insulin, insulin-like growth factor, hGH, tPA, cytokines, such as interleukines (IL), e.g. IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, interferon (IFN) alpha, IFN beta, IFN gamma, IFN omega or IFN tau, tumor necrosisfactor (TNF), such as TNF alpha and TNF beta, TNF gamma, TRAIL; G-CSF, GM-CSF, M-CSF, MCP-1 and VEGF. Also included is the production of erythropoietin or any other hormone growth factors. The method according to the invention can also be advantageously used for production of antibodies or fragments thereof. Such fragments include e.g. Fab fragments (Fragment antigen-binding=Fab). Fab fragments consist of the variable regions of both chains which are held together by the adjacent constant region. These may be formed by protease digestion, e.g. with papain, from conventional antibodies, but similar Fab fragments may also be produced in the mean time by genetic engineering. Further antibody fragments include F(ab′)2 fragments, which may be prepared by proteolytic cleaving with pepsin.

The protein of interest is preferably recovered from the culture medium as a secreted polypeptide, or it can be recovered from host cell lysates if expressed without a secretory signal. It is necessary to purify the protein of interest from other recombinant proteins and host cell proteins in a way that substantially homogenous preparations of the protein of interest are obtained. As a first step, cells and/or particulate cell debris are removed from the culture medium or lysate. The product of interest thereafter is purified from contaminant soluble proteins, polypeptides and nucleic acids, for example, by fractionation on immunoaffinity or ion-exchange columns, ethanol precipitation, reverse phase HPLC, Sephadex chromatography, chromatography on silica or on a cation exchange resin such as DEAE. In general, methods teaching a skilled person how to purify a protein heterologous expressed by host cells, are well known in the art.

Using genetic engineering methods it is possible to produce shortened antibody fragments which consist only of the variable regions of the heavy (VH) and of the light chain (VL). These are referred to as Fv fragments (Fragment variable=fragment of the variable part). Since these Fv-fragments lack the covalent bonding of the two chains by the cysteines of the constant chains, the Fv fragments are often stabilised. It is advantageous to link the variable regions of the heavy and of the light chain by a short peptide fragment, e.g. of 10 to 30 amino acids, preferably 15 amino acids. In this way a single peptide strand is obtained consisting of VH and VL, linked by a peptide linker. An antibody protein of this kind is known as a single-chain-Fv (scFv). Examples of scFv-antibody proteins of this kind are known from the prior art.

In recent years, various strategies have been developed for preparing scFv as a multimeric derivative. This is intended to lead, in particular, to recombinant antibodies with improved pharmacokinetic and biodistribution properties as well as with increased binding avidity. In order to achieve multimerisation of the scFv, scFv were prepared as fusion proteins with multimerisation domains. The multimerisation domains may be, e.g. the CH3 region of an IgG or coiled coil structure (helix structures) such as Leucin-zipper domains. However, there are also strategies in which the interaction between the VH/VL regions of the scFv are used for the multimerisation (e.g. dia-, tri- and pentabodies). By diabody the skilled person means a bivalent homodimeric scFv derivative. The shortening of the Linker in an scFv molecule to 5-10 amino acids leads to the formation of homodimers in which an inter-chain VH/VL-superimposition takes place. Diabodies may additionally be stabilised by the incorporation of disulphide bridges. Examples of diabody-antibody proteins are known from the prior art.

By minibody the skilled person means a bivalent, homodimeric scFv derivative. It consists of a fusion protein which contains the CH3 region of an immunoglobulin, preferably IgG, most preferably IgG1 as the dimerisation region which is connected to the scFv via a Hinge region (e.g. also from IgG1) and a Linker region. Examples of minibody-antibody proteins are known from the prior art.

By triabody the skilled person means a: trivalent homotrimeric scFv derivative. ScFv derivatives wherein VH-VL are fused directly without a linker sequence lead to the formation of trimers.

By “scaffold proteins” a skilled person means any functional domain of a protein that is coupled by genetic cloning or by co-translational processes with another protein or part of a protein that has another function.

The skilled person will also be familiar with so-called miniantibodies which have a bi-, tri- or tetravalent structure and are derived from scFv. The multimerisation is carried out by di-, tri- or tetrameric coiled coil structures.

By definition any sequences or genes introduced into a host cell are called “heterologous sequences” or “heterologous genes” or “transgenes” or “recombinant genes” with respect to the host cell, even if the introduced sequence or gene is identical to an endogenous sequence or gene in the host cell.

A sequence is called “heterologous sequence” even when the sequence of interest is the endogenous sequence but the sequence has been (artificially/intentionally/experimentally) brought into the cell and is therefore expressed from a locus in the host genome which differs from the endogenous gene locus.

A sequence is called “heterologous sequence” even when the sequence (e.g. cDNA) of interest is an (artificially/intentionally/experimentally) reintroduced (=recombinant) endogenous sequence and expression of this sequence is effected by an alteration/modification of a regulatory sequence, e.g. a promoter alteration or by any other means.

A “heterologous” protein is thus a protein expressed from a heterologous sequence.

Heterologous gene sequences can be introduced into a target cell by using an “expression vector”, preferably an eukaryotic, and even more preferably a mammalian expression vector. Methods used to construct vectors are well known to a person skilled in the art and described in various publications. In particular techniques for constructing suitable vectors, including a description of the functional components such as promoters, enhancers, termination and polyadenylation signals, selection markers, origins of replication, and splicing signals, are known in the prior art. Vectors may include but are not limited to plasmid vectors, phagemids, cosmids, artificial/mini-chromosomes (e.g. ACE), or viral vectors such as baculovirus, retrovirus, adenovirus, adeno-associated virus, herpes simplex virus, retroviruses, bacteriophages. The eukaryotic expression vectors will typically contain also prokaryotic sequences that facilitate the propagation of the vector in bacteria such as an origin of replication and antibiotic resistance genes for selection in bacteria. A variety of eukaryotic expression vectors, containing a cloning site into which a polynucleotide can be operatively linked, are well known in the art and some are commercially available from companies such as Stratagene, La Jolla, Calif.; Invitrogen, Carlsbad, Calif.; Promega, Madison, Wis. or BD Biosciences Clontech, Palo Alto, Calif.

In a preferred embodiment the expression vector comprises at least one nucleic acid sequence which is a regulatory sequence necessary for transcription and translation of nucleotide sequences that encode for a peptide/polypeptide/protein of interest.

The term “expression” as used herein refers to transcription and/or translation of a heterologous nucleic acid sequence within a host cell. The level of expression of a desired product/protein of interest in a host cell may be determined on the basis of either the amount of corresponding mRNA that is present in the cell, or the amount of the desired polypeptide/protein of interest encoded by the selected sequence as in the present examples. For example, mRNA transcribed from a selected sequence can be quantitated by Northern blot hybridization, ribonuclease RNA protection, in situ hybridization to cellular RNA or by PCR. Proteins encoded by a selected sequence can be quantitated by various methods, e.g. by ELISA, by Western blotting, by radioimmunoassays, by immunoprecipitation, by assaying for the biological activity of the protein, by immunostaining of the protein followed by FACS analysis or by homogeneous time-resolved fluorescence (HTRF) assays.

In the present invention the term “expression” is equally used in the context of a gene, meaning the DNA sequence, as well as in the context of a protein product into which the DNA sequence is translated. The terms “gene” and “protein” can thus be used interchangeably in the context of expression, e.g. “expression of a protein of interest” and “expression of a gene of interest” are used interchangeably and both wordings refer to the same matter of fact. In the present invention, these terms refer preferredly to human genes and proteins, but included are equally homologous sequences from other mammalian species, preferredly mouse, hamster and rat, as well as homologous sequences from additional eucaryotic species including chicken, duck, moss, worm, fly and yeast.

The term “effecting” the expression or “effecting” the secretion of a protein of interest as used herein refers to positively influencing the same or causing the same. These terms as used herein preferably refer to “increasing the expression” or “increasing the secretion”.

“Transfection” of eukaryotic host cells with a polynucleotide or expression vector, resulting in genetically modified cells or transgenic cells, can be performed by any method well known in the art. Transfection methods include but are not limited to liposome-mediated transfection, calcium phosphate co-precipitation, electroporation, polycation (such as DEAE-dextran)-mediated transfection, protoplast fusion, viral infections and microinjection. Preferably, the transfection is a stable transfection. The transfection method that provides optimal transfection frequency and expression of the heterologous genes in the particular host cell line and type is favoured. Suitable methods can be determined by routine procedures. For stable transfectants the constructs are either integrated into the host cell's genome or an artificial chromosome/mini-chromosome or located episomally so as to be stably maintained within the host cell.

The practice of the present invention will employ, unless otherwise indicated, conventional techniques of cell biology, molecular biology, cell culture, immunology and the like which are in the skill of one in the art. These techniques are fully disclosed in the current literature.

The invention relates to a method of producing a heterologous protein of interest in a cell comprising a) increasing the expression of at least one gene encoding a SM-protein or the activity of the respective protein or at least one derivative, mutant or fragment thereof, and b) effecting the expression of said heterologous protein of interest.

The invention specifically relates to a method of producing a heterologous protein of interest in a cell comprising a) increasing the expression of at least one gene encoding a protein from the SEC1/Munc18 group of proteins (SM-protein), and b) effecting the expression of said heterologous protein of interest. Preferably the secretion of the protein of interest in method step b) is increased. The invention thus preferably relates to a method of producing a heterologous protein of interest in a cell comprising a) increasing the expression of at least one gene encoding a protein from the SEC1/Munc18 group of proteins (SM-proteins), and b) increasing the secretion of said heterologous protein of interest.

The invention preferably relates to a method of producing a heterologous protein of interest in a cell comprising a) increasing the expression of at least one gene encoding a protein selected from the SEC1/Munc18 group of proteins (SM-proteins) consisting of:

Sec1p, Sly1 p, Vps33p and Vps45p, ROP, Sly1 and Vps33/carnation, Unc-18, Munc18-1, 2 and -3, VPS45, VPS33-A, VPS33-B and Sly1,
and b) effecting the expression of said heterologous protein of interest, preferably increasing the expression or particularly preferred the secretion of said heterologous protein of interest.

Preferably the protein in step a) is selected from the SEC1/Munc 18 group of proteins (SM-proteins) consisting of: Sec1p, Sly1 p, Vps33p, Vps45p, Munc18-1, -2 and -3, VPS45, VPS33-A and -B and Sly1.

More preferred the protein in step a) is selected from the SEC1/Munc18 group of proteins (SM-proteins) consisting of: Munc18-1, -2 and -3, VPS45, VPS33-A and -B and Sly1. Most preferred the protein in step a) is selected from the SEC1/Munc18 group of proteins (SM-proteins) consisting of: Munc 18-3/Munc 18c and Sly-1 .

In a specific embodiment of the present invention the method is characterized in that one gene in step a) encodes a Munc-18 protein or a Munc-18 protein family member. In a specific embodiment of the present invention the method is characterized in that one gene in step a) encodes one of the three Munc18 isoforms, Munc18a, b or c, preferably Munc 18c.

In another specific embodiment of the present invention the method is characterized in that one gene in step a) encodes Munc18c (SEQ ID NO: 39).

In a specific embodiment of the present invention the method is characterized in that one gene in step a) encodes a Sly-1 protein or a Sly-1 protein family member, preferably Sly-1.

In a further specific embodiment of the present invention the method is characterized in that one gene in step a) encodes Sly-1 (SEQ ID NO: 41).

In a preferred embodiment of the present invention the method is characterized in that step a) comprises increasing the expression or activity of at least two genes encoding SM-proteins, whereby said SM proteins are involved in two different steps of vesicle transport.

In a specific embodiment of the present invention the method is characterized in that a) one gene encodes a SM protein, which regulates the fusion of vesicles with the plasma membrane, b) the second gene encodes a SM protein, which regulates the fusion of vesicles with the Golgi complex.

In a specifically preferred embodiment of the present invention the method is characterized in that the expression or activity of Munc18c (SEQ ID NO: 39) and Sly-1 (SEQ ID NO: 41) is increased.

In a further embodiment of the present invention the method is characterized in that step a) comprises a) increasing the expression or activity of a first gene encoding a member of the SM protein family, b) a second gene encoding another member of the SM protein family, and c) a third gene encoding XBP-1.

In a specifically preferred embodiment of the present invention the method is characterized in that the expression or activity of Munc18c (SEQ ID NO: 39), Sly-1 (SEQ ID NO: 41), and XBP-1 (SEQ ID NO: 43) is increased.

The invention furthermore relates to a method of engineering a cell comprising a) introducing into a cell one or more vector systems comprising nucleic acid sequences encoding for at least two polypeptides whereby i) at least one first nucleic acid sequence encodes a SM-protein or a derivative, mutant or fragment thereof, and ii) a second nucleic acid sequence encodes a protein of interest b) expressing said protein of interest and said at least one SM-protein or a derivative, mutant or fragment thereof in said cell.

In a specific embodiment of the present invention the method is characterized in that the nucleic acid sequences are sequentially introduced into said cell.

In a further specific embodiment of the present invention the method is characterized in that at least one nucleic acid sequences encoding a SM protein is introduced before the nucleic acid sequence encoding said protein of interest.

In another embodiment of the present invention the method is characterized in that at least one nucleic acid sequences encoding a protein of interest is introduced before the nucleic acid sequence encoding said SM protein.

In a preferred embodiment of the present invention the method is characterized in that the nucleic acid sequences are simultaneously introduced into said cell.

In a specific embodiment of the present invention the method is characterized in that the SM-protein is either one of the Munc-18 isoforms, preferably Munc-18c (SEQ ID NO: 39), or Sly-1 (SEQ ID NO: 41).

In a preferred embodiment of the present invention the method is characterized in that in step a) i) two SM-proteins are used in combination, whereby said SM proteins are involved in two different steps of vesicle transport.

In a further embodiment of the present invention the method is characterized in that a) one gene encodes a SM protein, which regulates the fusion of vesicles with the plasma membrane, b) the second gene encodes a SM protein, which regulates the fusion of vesicles with the Golgi complex.

In a specific embodiment of the present invention the method is characterized in that the two SM-proteins used in combination are Munc-18c (SEQ ID NO: 39) and Sly-1 (SEQ ID NO: 41).

In a preferred embodiment of the present invention the method is characterized in that in step a) i) two SM-proteins are used in combination with XBP-1.

In a specifically preferred embodiment of the present invention the method is characterized in that the SM proteins are Munc-18c (SEQ ID NO: 39) and Sly-1 (SEQ ID NO: 41) in combination with XBP-1 (SEQ ID NO: 43).

In another embodiment of the present invention the method is characterized in that said cell is a eukaryotic cell such as a yeast, plant, worm, insect, avian, fish, reptile or mammalian cell.

In a specific embodiment of the present invention the method is characterized in that said cell is a eukaryotic cell, preferably a vertebrate cell, most preferably a mammalian cell.

Preferrably, said vertebrate cell is an avian cell, such as a chicken or duck cell.

In a further specific embodiment of the present invention the method is characterized in that said mammalian cell is a Chinese Hamster Ovary (CHO), monkey kidney CV1, monkey kidney COS, human lens epithelium PER.C6™, human embryonic kidney HEK293, human myeloma, human amniocyte, baby hamster kidney, African green monkey kidney, human cervical carcinoma, canine kidney, buffalo rat liver, human lung, human liver, mouse mammary tumor or myeloma cell, NSO, a dog, pig or macaque cell, rat, rabbit, cat, goat, preferably a CHO cell.

In a preferred embodiment of the present invention the method is characterized in that said CHO cell is CHO wild type, CHO K1, CHO DG44, CHO DUKX-B11, CHO Pro-5 or mutants derived thereof, including the CHO mutants Lec1 to Lec35, preferably CHO DG44.

In a further embodiment of the present invention the method is characterized in that the protein of interest is a therapeutic protein.

In a specific embodiment of the present invention the method is characterized in that the protein of interest is a membrane or secreted protein, preferably an antibody or antibody fragment.

In a further specific embodiment of the present invention the method is characterized in that the antibody is monoclonal, polyclonal, mammalian, murine, chimeric, humanized, primatized, primate, human or an antibody fragment or derivative thereof such as antibody, immunoglobulin light chain, immunoglobulin heavy chain, immunoglobulin light and heavy chains, Fab, F(ab′)2, Fc, Fc-Fc fusion proteins, Fv, single chain Fv, single domain Fv, tetravalent single chain Fv, disulfide-linked Fv, domain deleted, minibody, diabody, or a fusion polypeptide of one of the above fragments with another peptide or polypeptide, Fc-peptide fusion, Fc-toxine fusion, scaffold proteins.

In a further embodiment of the present invention the method is characterized in that said heterologous SM protein is present in the vesicle fusion complex comprising at least one SNARE protein.

In a specific embodiment of the present invention the method is characterized in that said heterologous SM protein is present in the vesicle fusion complex comprising at least one SNARE protein and Syntaxin 4 or Syntaxin 5.

In a further embodiment of the present invention the method is characterized in that the specific productivity of said heterologous protein of interest in said cell is at least 5 pg per cell and day, 15 pg per cell and day, 20 pg per cell and day, 25 pg per cell and day.

In another embodiment of the present invention the method is characterized in that said method results in increased specific cellular productivity of said protein of interest in said cell in comparison to a control cell expressing said protein of interest, but whereby said control cell does not have increased expression or activity of any SM-protein.

In a preferred embodiment of the present invention the method is characterized in that the increase in productivity is about 5% to about 10%, about 11% to about 20%, about 21% to about 30%, about 31% to about 40%, about 41% to about 50%, about 51% to about 60%, about 61% to about 70%, about 71% to about 80%, about 81% to about 90%, about 91% to about 100%, about 101% to about 149%, about 150% to about 199%, about 200% to about 299%, about 300% to about 499%, or about 500% to about 1000%.

The invention furthermore relates to a method of increasing specific cellular productivity or the titer of a membrane or secreted protein of interest in a cell comprising a) introducing into a cell one or more vector systems comprising nucleic acid sequences encoding for at least two polypeptides whereby i) at least one first polynucleotide encodes a SM-protein or a derivative, mutant or fragment thereof, and ii) a second polynucleotide encodes a protein of interest and b) expressing said protein of interest and said SM-protein or a derivative, mutant or fragment thereof in said cell.

The invention furthermore relates to an expression vector comprising expression units encoding at least two polypeptides, whereby a) at least one polypeptide is a SM-protein or a derivative, mutant or fragment thereof, and b) a second polypeptide is a protein of interest.

In a specific embodiment of the present invention the expression vector is characterized in that the protein of interest is a therapeutic protein, preferably an antibody or antibody fragment.

In a preferred embodiment of the present invention the expression vector is characterized in that the antibody is monoclonal, polyclonal, mammalian, murine, chimeric, humanized, primatized, primate, human or an antibody fragment or derivative thereof such as antibody, immunoglobulin light chain, immunoglobulin heavy chain, immunoglobulin light and heavy chains, Fab, F(ab′)2, Fc, Fc-Fc fusion proteins, Fv, single chain Fv, single domain Fv, tetravalent single chain Fv, disulfide-linked Fv, domain deleted, minibody, diabody, or a fusion polypeptide of one of the above fragments with another peptide or polypeptide, Fc-peptide fusion, Fc-toxine fusion, scaffold proteins.

In another embodiment of the present invention the expression vector is characterized in that the expression units are multicistronic, preferably bicistronic.

In a specific embodiment of the present invention the expression vector is characterized in that the vector comprises any of the expression constructs described in FIG. 8.

In a preferred embodiment of the present invention the expression vector is characterized in that the vector comprises at least one bicistronic expression unit arranged as follows a) a gene encoding a SM protein, b) an IRES element and c) a second gene encoding a SM protein. See FIG. 8 d).

In another preferred embodiment of the present invention the expression vector is characterized in that it encodes at least one protein of interest (GOI) and one SM protein from separate expression units (FIG. 8 a) or from one bi-cistronic unit (FIG. 8 b). In further preferred embodiment of the present invention the expression vector is characterized in that it comprises genes of two SM proteins encoded either from separate expression cassettes (FIG. 8 c) or bi-cistronically, whereby the two genes are linked via an IRES element (FIG. 8 d). In a further embodiment of the present invention the expression vector is characterized in that it encodes at least two SM proteins and a gene of interest (FIG. 8 e) or several SM proteins from one multi-cistronic expression unit.

In a preferred embodiment of the present invention the expression vector is characterized in that the SM-protein is one of the Munc-18 isoforms Munc a, b, c, preferably Munc-18c (SEQ ID NO: 39).

In a further preferred embodiment of the present invention the expression vector is characterized in that the SM-protein is Sly-1 (SEQ ID NO: 41).

In a further embodiment of the present invention the expression vector is characterized in that at least two SM-proteins are used in combination.

In a specific embodiment of the present invention the expression vector is characterized in that said at least two SM proteins are involved in two different steps of vesicle transport.

In another embodiment of the present invention the expression vector is characterized in that a) one SM protein regulates the fusion of vesicles with the plasma membrane, b) the second SM protein regulates the fusion of vesicles with the Golgi complex.

In a preferred embodiment of the present invention the expression vector is characterized in that the SM proteins are Munc-18c (SEQ ID NO: 39) and Sly-1 (SEQ ID NO: 41).

In a further preferred embodiment of the present invention the expression vector is characterized in that at least two SM-proteins are used in combination with XBP-1, preferably Munc-18c (SEQ ID NO: 39) and Sly-1 (SEQ ID NO: 41) in combination with XBP-1 (SEQ ID NO: 43).

The invention furthermore relates to a cell expressing at least two heterologous genes: a) at least one gene encoding a SM-protein or a derivative, mutant or fragment thereof and b) a gene encoding a protein of interest.

In a specific embodiment of the present invention the cell is characterized in that the protein of interest is a therapeutic protein, preferably an antibody or antibody fragment.

In a preferred embodiment of the present invention the cell is characterized in that the antibody is monoclonal, polyclonal, mammalian, murine, chimeric, humanized, primatized, primate, human or an antibody fragment or derivative thereof such as antibody, immunoglobulin light chain, immunoglobulin heavy chain, immunoglobulin light and heavy chains, Fab, F(ab′)2, Fc, Fc-Fc fusion proteins, Fv, single chain Fv, single domain Fv, tetravalent single chain Fv, disulfide-linked Fv, domain deleted, minibody, diabody, or a fusion polypeptide of one of the above fragments with another peptide or polypeptide, Fc-peptide fusion, Fc-toxine fusion, scaffold proteins.

In a specific embodiment of the present invention the cell is characterized in that the expression level of the SM protein is significantly above the endogenous level, preferably 10%. In another embodiment of the present invention the cell is characterized in that the expression level of said protein is 5% above the endogenous level, preferably 10%, 15%, 20%, 25%, 30%, 35%, 40%, 45%, 50%, 55%, 60%, 65%, 70%, 75%, 80%, 85%, 90%, 95%, 100%, 120%, 150%, 175%, 200%, 300%, 400%, 500%, 1000% above the endogenous level.

In a further embodiment of the present invention the cell comprises any of the expression vectors of the present invention.

In a specific embodiment of the present invention the cell is characterized in that said cell is a eukaryotic cell, preferably a vertebrate cell, most preferably a mammalian cell. Specifically preferred is a rodent cell.

In a preferred embodiment of the present invention the cell is characterized in that said eukaryotic cell is an avian cell.

In a further specific embodiment of the present invention the cell is characterized in that said mammalian cell is a rodent cell, preferably a hamster or murine cell. In a preferred embodiment of the present invention the cell is characterized in that said mammalian cell is a Chinese Hamster Ovary (CHO), monkey kidney CV1, monkey kidney COS, human lens epithelium PER.C6™, human myeloma, human amniocyte, human embryonic kidney, HEK 293, baby hamster kidney, African green monkey kidney, human cervical carcinoma, canine kidney, buffalo rat liver, human lung, human liver, mouse mammary tumor or myeloma cell, NSO, a dog, pig or macaque cell, rat, rabbit, cat, goat, preferably a CHO cell.

In a further preferred embodiment of the present invention the cell is characterized in that said CHO cell is CHO wild type, CHO K1, CHO DG44, CHO DUKX-B11, CHO Pro-5 or mutants derived thereof, including the CHO mutants Lec1 to Lec35, preferably CHO DG44.

In a specifically preferred embodiment of the present invention the cell is characterized in that said cell is a CHO cell, preferrably a CHO DG44 cell.

The invention furthermore relates to a protein of interest, preferably an antibody produced by any of the methods of the present invention.

The invention further relates to a pharmaceutical composition comprising a compound useful for blocking or reducing the activity or expression, preferably the expression, of one or several SM-proteins and a pharmaceutically acceptable carrier.

In a specific embodiment of the present invention the pharmaceutical composition is characterized in that the compound is a polynucleotide sequence. Preferrably, the polynucleotide sequence is shRNA, RNAi, siRNA or antisense-RNA, most preferably shRNA.

In a further specific embodiment of the present invention the pharmaceutical composition is characterized in that the SM-protein is Munc-18c (SEQ ID NO: 39) or Sly-1 (SEQ ID NO: 41) or a combination of the two.

The invention furthermore relates to a method for identifying a modulator of SM-protein function comprising a) providing at least a SM-protein or a derivative, mutant or fragment thereof, preferably Munc-18c, b) contacting said SM-protein of step a) with a test agent, c) determining an effect related to increased or decreased protein secretion or expression of cell-surface proteins.

The invention further relates to a method for the treatment of cancer, auto-immune diseases and inflammation comprising, administering to a patient in need thereof a therapeutically effective amount of a pharmaceutical composition according to the invention.

The invention also relates to a method comprising application of a pharmaceutical composition according to the present invention for the treatment of cancer, auto-immune diseases and inflammation.

The invention also relates to a method of inhibiting or reducing the proliferation or migration of a cell comprising contacting said cell with a pharmaceutical composition according to the invention.

Possible therapeutic applications of the present invention include preventing secretion of proteins such as inflammatory mediators, growth factors, angiogenic factors from cells or tissues in order to control cell-cell communication in cancer therapy, auto-immune diseases and inflammation, or reduction of cell attachment by reducing cell-surface presence of anchoring transmembrane-proteins for the purpose of facilitating growth in suspension and preventing cell aggregation.

The invention furthermore relates to the use of a SM-protein or a polynucleotide encoding a SM-protein in an in vitro cell or tissue culture system to increase secretion and/or production of a protein of interest. Preferably the SM protein is a Munc 18 protein such as Munc18c (SEQ ID NO: 39). Also preferred is a Sly-1 protein such as Sly-1 (SEQ ID NO: 41).

The invention further relates to a diagnostic use of any of the methods, expression vectors, cells or pharmaceutical compositions of the present invention.

The invention additionally relates to a method for enhancing the protein secretion of a cell/engineering a cell/producing a heterologous protein of interest in a cell comprising

a) cloning of human Sec1/Munc18 and Sly1/SCFD1 into expression vectors (e.g. the mammalian BI-HEX® expression platform), whereby said proteins can be encoded by one or different bi-/multi-cistronic expression units and whereby said proteins can be contained on the same or on different plasmids,
b) transfection of said constructs, either alone or in combination, either simultaneously or sequentially, into eukaryotic host cells, preferredly mammalian cells such as CHO, BHK, NSO, HEK293, PerC.6,
c) optionally: verification of transgene expression,
d) introduction of a construct encoding a gene-of-interest (GOI), preferredly a secreted or transmembrane protein,
e) expression analysis of the GOI, e.g. by ELISA, Western Blot or flow-cytometry.

Alternatively, the order of the steps (b+c) and (d+e) can be changed, thereby introducing the GOI first, or the steps (b) and (d) can be done simultaneously.

The invention generally described above will be more readily understood by reference to the following examples, which are hereby included merely for the purpose of illustration of certain embodiments of the present invention. The following examples are not limiting. They merely show possible embodiments of the invention. A person skilled in the art could easily adjust the conditions to apply it to other embodiments.

### Experimental

### Materials and Methods

### Plasmid Design.

Human sly1 is RT-PCR-amplified from HEK-293 total RNA using oligonucleotides ORP70 (5′-CGCGGATCCACCATGGCGGCGGCGGCGGCAGCG-3′, SEQ ID NO 1) and ORP71 (5′-CCGCTCGAGTTACTTTTGTCCAAGTTGTGACAACTG-3′, SEQ ID NO 2, and cloned BamHI/XhoI into pcDNA3.1 (Invitrogen) to result in pRP24 (P_{hCMV}-sly1-pA_{SV40}). Likewise, munc18c is cloned (ORP69, 5′-CGCGGATCCACCATGGCGCCGCCGGTGGCAGAGAGG-3′, SEQ ID NO 3; ORP66, 5′-CCCTCGAGCTATTCATCTTTAATTAAGGAGAC-3′, SEQ ID NO 4), which results in pRP17 (P_{hCMV}-munc18c-pA_{SV40}). pRP32 (P_{hCMV}-EYFP-sly1-pA_{SV40}) is constructed by inserting sly1, PCR-amplified from pRP24 using ORP29 (5′-CTCAGATCTGCGGCGGCGG CGGCAGCG-3′, SEQ ID NO 5) and ORP30 (5′-ACCGTCGACCTTTTGTCCAAGTTGTGACAACTG-3′, SEQ ID NO 6), Bg1II/Sa1I into pEYFP-C1 (Clontech). pRP23 (P_{hCMV}-EYFP-munc18c-pASV40) is designed by excising munc18c BamHI/XhoI from pRP17 and cloning it Bg1II/Sa1I into pEYFP-C1. pRP3 is generated by inserting sly1, PCR-amplified using ORP9 (5′-CGCGCGGCCGCAC CATGGCGGCGGCGGCGGCAGCG-3′, SEQ ID NO 7) and ORP10 (5′-CCGGGATCCTTACTTTTGTCC AAGTTGTGACAACTG-3′, SEQ ID NO 8), NotI/BamHI into pRP1, derived from pIRESneo (Clontech) by replacing the neomycin resistance-conferring gene with SmaI/XbaI GFP, PCR-amplified from pLEGFP-N1 (Clontech) using ORP5 (5′-CCCCCGGGATGGTGAGCAAGGGCGAGG-3′, SEQ ID NO 9) and ORP6 (5′-TTTCTAGATTACTTGTACAGCTCGTCC-3′, SEQ ID NO 10). Likewise, pRP4 is constructed by inserting the munc18c, PCR-amplified from pRP17 (ORP15, 5′-C GCGCGGCCGCACCATGGCGCCGCCGGTGGCAGAGAGG-3′, SEQ ID NO 11; ORP16, 5′-CCGGATC CCTATTCATCTTTAATTAAGGAGAC-3′, SEQ ID NO 12) NotI/BamHI into pRP1. pRP29 (P_{hCMV}-ECFP-syntaxin4-pA_{SV40}) is constructed by PCR-mediated amplification of syntaxin 4 (ORP127, 5′-CCCAAGCTTTGCGCGACAGGACCCACGAG-3′, SEQ ID NO 13; ORP128, 5′-CGCGTCGACTTATC CAACGGTTATGGTGATGCC-3′, SEQ ID NO 14) followed by cloning HindIII/Sa1I into pECFP-C1 (Clontech). Likewise, syntaxin 5 is cloned (ORP136, 5′-GGAAGATCTATCCCGCGGA AACGCTAC-3′, SEQ ID NO 15; ORP137, 5′-CCCAAGCTTTCAAGCAAGGAAGACCAC-3′, SEQ ID NO 16), which results in pRP40 (P_{hCMV}-ECFP-syntaxin5-pA_{SV40}). Expression vectors harboring sly1- or munc18c-specific shRNAs are cloned by inserting double-stranded DNA-fragments BbsI/XbaI into pmU6: (i) sly1 (shRNA_{sly1}_{_{—}}₁; pRP5, 5′-TTTGGAAGTAAACTGGAAGAT ATTTTCAAGAGAAATATCTTCCAGTTTACTTCTTTTT-3′, SEQ ID NO 23, and 5′-CTAGAAAAAGAAGTAAACTGGAAGATATTTCTCTTGAAAATATCTTCCAGTTT ACTTC-3′; SEQ ID NO 24, shRNA_{sly1}_{_{—}}₂; pRP6, 5′-TTTGGCAGTGAAACTAGACAAGAAATTCAAGAGATTTCTTGTCTAGTTTCACTG CTTTTT-3′, SEQ ID NO 25 and 5′-CTAGAAAAAGCAGTGAAACTAGACAAGAAATCTCTTGAATTTCTTGTCTAGTT TCACTGC-3′; SEQ ID NO 26 shRNA_{sly1}_{_{—}}₃; pRP7, 5′-TTTGGGAGGCAACTAC ATTGAATATTTCAAGAGAATATTCAATGTAGTTGCCTCCTTTTT-3′, SEQ ID NO 27, and 5′-CTAGAAAAAGGAGGCAACTACATTGAATATTCTCTTGAAATATT CAATGTAGTTGCCTCC-3′, SEQ ID NO 28); (ii) munc18c (shRNA_{munc18c}_{_{—}}₁; pRP12, 5′-TTTGCACATGAATCTCAGGTGTATATTCAAGAGATATACACCTGAGATTCATGT GTTTTT-3′, SEQ ID NO 29, and 5′-CTAGAAAAACACATGA ATCTCAGGTGTATATCTCTTGAATATACACCTGAGATTCATGTG-3′, SEQ ID NO 30; shRNA_{munc18c}_{_{—}}₂; pRP14, 5′-TTTGGCTTGAAGACTACTACAAGATTTCAAG AGAATCTTGTAGTAGTCT TCAAGCTTTTT-3′, SEQ ID NO 31, and 5′-CTAGAAAAAGCTTGAAGACTACTACAAGATTCTCTTGAAATCTTGTAGTAGTCT TCAAGC-3′, SEQ ID NO 32; shRNA_{munc18c}_{_{—}}₃; pRP38, 5′-TTTGCGCCAGAAAC CCAGAGCTAATTTCAAGAGAATTAGCTCTGGGTTTCTGGCGTTTTT-3′, SEQ ID NO 33, and 5′-CTAGAAAAACGCCAGAAACCCAGAGCTAATTCTCTTGAAATT AGCTCTGGGTTTCTGG CG-3′, SEQ ID NO 34; shRNA_{munc18c}_{_{—}}₄; pRP39, 5′-TTTGGCTGAATAAACCCAAGGATAATTCAAGAGATTATCCTTGGGTTTATTCAG CTTTTT-3′, SEQ ID NO 35, and 5′-CTAGAAAAAGCTGAATAAACCCA AGGATAATCTCTTGAATTATCCTTGGGTTTATTCAGC-3′, SEQ ID NO 36); (iii) Control shRNA (pRP9,5′-TTTGCACAAGCTGGAGTACAACTACTTCAAGAG AGTAGTTGTACTCCAGCTT GTGTTTTT-3′, SEQ ID NO 37, and 5′-CTAGAAAAACACAAGCTGGAGTACAACTACTCTCTTGAAGTAGTTGTACTCCA GCTTGTG-3′, SEQ ID NO 38).

pSEAP2-control encoding the human placental alkaline phosphatase (SEAP) is purchased from Clontech and pSS158 harboring the Bacillus stearothermophilus-derived secreted α-amylase (SAMY) has been described before 49. pWW276 containing human vascular endothelial growth factor 121 (VGEF121) as well as pWW943 and pWW946 encoding heavy and light chains of the human IgG1 Rituximab, respectively, are kindly provided by Wilfried Weber. The xbp-1 expression vector pcDNA3.1-Xbp-1 (P_{hCMV}-xbp-1-pASV40) has been described before (Tigges and Fussenegger, 2006).

### Cell Culture and Transfection

### a) Cultivation of Adherent Cells:

Chinese hamster ovary (CHO-K1; ATCC CCL-61) and human embryonic kidney cells (HEK-293; ATCC CRL-1573) are cultivated in ChoMaster HTS medium (Cell Culture Technology, Gravesano, Switzerland) or Dulbecco's modified Eagle's medium (DMEM; Invitrogen, Carlsbad, Calif., USA) supplemented with 5% FCS (PAN Biotech, Aidenbach, Germany; cat. no. 3302, lot no. P231902) at 37° C. in a humidified atmosphere containing 5% CO₂. For transient transfection, 1×10⁵ cells are seeded into one well of a 12-well tissue culture plate and transfected after 24 h using a modified calcium phosphate-based protocol⁴⁷ or the FuGENE6 transfection reagent (Roche, Basel, Switzerland). Monotransgenic stable CHO-K1 derivatives engineered for constitutive transgene expression are produced using the following combinations of expression and selection vectors as well as antibiotics: (i) CHO-Sly1₁₆ and CHO-Sly1₂₃; pRP24; 400 μg/ml G418 (Merck); (ii) CHO-Munc18c₈ and CHO-Munc18c₉, pRP17; 400 μg/ml G418. Double-transgenic cell lines CHO-Sly1-Munc18c₁ and CHO-Sly1-Xbp1₄ are constructed by co-transfection of pRP17 and pPUR (Clontech), pcDNA3.1-Xbp-1³⁵ and pPUR, respectively, into CHO-Sly1₂₃ followed by clonal selection with G418 and puromycin (4 μg/ml). The triple-transgenic cell line CHO-Sly1-Munc18c-Xbp-1₇ enabling constitutive expression of sly1, munc18c and xbp-1, is generated by co-transfection of pcDNA3.1-Xbp-1 and pZeoSV2 (Invitrogen) into CHO-Sly1-Munc18c₁ followed by selection with G418 (400 μg/ml), puromycin (4 μg/ml) and zeocin (150 μg/ml).

### b) Suspension Cultures

Suspension cultures of monoclonal antibody (mAB) producing CHO-DG44 cells (Urlaub et al., 1986) and stable transfectants thereof are incubated in a BI proprietary chemically defined, serum-free media. Seed stock cultures are sub-cultivated every 2-3 days with seeding densities of 3×10⁵−2×10⁵ cells/mL respectively. Cells are grown in T-flasks or shake flasks (Nunc). T-flasks are incubated in humidified incubators (Thermo) and shake flasks in Multitron HT incubators (Infors) at 5% CO₂, 37° C. and 120 rpm.

The cell concentration and viability is determined by trypan blue exclusion using a hemocytometer.

### Fed Batch Cultivation

Cells are seeded at 3×10⁵ cells/ml into 1000 ml shake flasks in 250 ml of BI-proprietary production medium without antibiotics or MTX (Sigma-Aldrich, Germany). The cultures are agitated at 120 rpm in 37° C. and 5% CO₂ which is later reduced to 2% as cell numbers increase. Culture parameters including pH, glucose and lactate concentrations are determined daily and pH is adjusted to pH 7.0 using NaCO₃ as needed. BI-proprietary feed solution is added every 24 hrs. Cell densities and viability are determined by trypan-blue exclusion using an automated CEDEX cell quantification system (Innovatis). Samples from the cell culture fluid are collected at and subjected to titer measurement by ELISA.

For ELISA antibodies against human-Fc fragment (Jackson Immuno Research Laboratories) and human kappa light chain HRP conjugated (Sigma) are used.

Cumulative specific productivity is calculated as product concentration at the given day divided by the “integral of viable cells” (IVC) until that time point.

### RNA Isolation, RT-PCR and Quantitative Real-Time PCR

Total RNA is prepared from mammalian cells using NucleoSpin RNA II kit (Macherey-Nagel, Oensingen, Switzerland) and RT-PCR is performed with the TITANIUM™ One-Step RT-PCR kit (Clontech) according to the manufacturer's protocol. Relative quantification of seap, samy and vegf₁₂₁ mRNA is performed with an Applied Biosystems 7500 real-time PCR device using 25 μl reactions containing Power SYBR Green PCR Master Mix (Applied Biosystems, Warrington, UK), 100 ng of cDNA, 900 nM of a forward and reverse primers specific for seap (5′-AGGCCCGGGACAGGAA-3′, SEQ ID NO 17; 5′-GCCGTCCTTGAGCACATAGC-3′, SEQ ID NO 18), samy (5′-AAA GCTCAATATCTTCAAGCCATTC-3′, SEQ ID NO 19; 5′-AACACGACATCGGCGTACACT-3′, SEQ ID NO 20) and vegf₁₂₁ (5′-CTTGCTGCTCTACCTCCACCAT-3′, SEQ ID NO 21; 5′-TGATTCTGCCCTCCTCCT TCT-3′, SEQ ID NO 22). All samples are standardized using a ribosome 18s-RNA-specific transcript assay (Applied Biosystems) and melting curve analysis is conducted for all amplicons to confirm the absence of non-specific amplification.

### Confocal Microscopy

HEK-293, seeded and transfected on poly-lysine-coated glass slides are washed after 48 h with phosphate-buffered saline (PBS), fixed with paraformaldehyde (3% w/v), washed again with PBS again and analysed by confocal microscopy. Images are recorded with a Leica TCS SPI (Leica, Heerbrugg, Switzerland) and analyzed by Adobe Photoshop 10.

### Antibodies, Immunoprecipitation and Western Blot

Mammalian cells are lysed on ice in lysis buffer (50 mM Tris-HCL, pH 7.5, 150 mM NaCl, 1 mM DTT, 1 mM EDTA, 1% Triton X-100). Total protein lysates are obtained by centrifugation at 14,000×g for 10 min at 4° C. followed by incubation with Protein A-Sepharose beads (Amersham Biosicences, Uppsla, Sweden) for 30 min at 4° C. Immunoprecipitation is performed by mixing 2 mg of total protein with affinity-purified Munc18c antibodies coupled to Protein A-Sepharose in a final volume of 500 μl lysis buffer by rotation at 4° C. overnight. The beads are then washed four times with 500 μl lysis buffer and the protein is eluted and separated by SDS-PAGE followed by Western blotting analysis. Antibodies specific for Sly1 are kindly provided by Jesse Hay (University of Montana, Missoula, Mo., USA). Antibodies specific for Munc18a, syntaxin 4 and Vamp2 are purchased from Synaptic Systems (Goettingen, Germany) and antibodies against Munc18b, Munc18c and p27^{Kip1} are from Santa Cruz Biotechnology (Santa Cruz, Calif., USA). Blotted protein is visualized using ECL-Plus detection reagents and HRP-conjugated secondary antibodies (Amersham, Piscataway, N. J., USA).

### Protein Production

Protein production is assessed after 48 h in culture using standardized assays: SEAP, p-nitrophenylphosphate-based light-absorbance time course; SAMY, blue starch Phadebas® assay (Pharmacia Upjohn, Peapack, N.J., cat. no. 10-5380-32); VEGF₁₂₁, by the human VEGF₁₂₁-specific ELISA (R&D Systems, Minneapolis, Minn., cat. no. DY293) and Rituximab by ELISA (Sigma, cat. no. I2136 and A0170).

Antibody titer and specific productivity of cells growing in suspension cultures is determined as follows:

Antibody producing CHO-DG44 are transfected with bicistronic vectors to analyse the effect of heterologous protein expression on mAb productivity. To assess the productivity in seed stock culture, samples from cell culture supernatant are collected from three consecutive passages. The product concentration is then analysed by enzyme linked immunosorbent assay (ELISA). For ELISA antibodies against human-Fc fragment (Jackson Immuno Research Laboratories) and human kappa light chain HRP conjugated (Sigma) are used. Together with the cell densities and viabilities the specific productivity can be calculated as follows:

$$qp = \frac{\frac{( {mAb}_{P + 1} + {mAb}_{P} )}{2}}{( {t}_{P + 1} - {t}_{P} ) ⋆ ( \frac{{cc}_{P + 1} + {cc}_{p}}{2} )}$$

$$qp = specific productivity ( pg \text{/} cell \text{/} day )$$

$$mAb = antibody concentration ( mg \text{/} L )$$

$$t = time point ( days )$$

$$cc = cell count ( \times {10}^{6} cells / mL )$$

$$P = passage$$

### N-Linked Glycosylation Profile of Rituximab

Rituximab is purified using protein A-Sepharose and eluted with 10 mM glycine buffer (pH 2.8), followed by neutralization with 2M Tris, pH9.0. The purity/integrity is confirmed by SDS-PAGE. Oligosaccharides are then enzymatically released from the antibodies by N-Glycosidase digestion (PNGaseF, EC 3.5.1.52, QA-Bio, San Mateo, Calif.) at 0.05 mU/mg protein in 2 mM Tris, pH7 for 3 h at 37° C. The released oligosaccharides are incubated in 150 mM acetic acid prior to the MALDI analysis with DHB as matrix (Papac et al., 1998) using an Autoflex MALDI/TOF (Bruker Daltonics, Faellanden, Switzerland) operating in positive ion mode.

### HRP Transport Assay

Human HT1080 fibrosarcoma cells are co-transfected with constructs encoding secreted horseraddish peroxidase (ssHRP) and either empty vector, expression constructs for Munc18c, Sly1 or a bi-cistronic expression unit encoding both Munc18c and Sly1. After 24 h and 48 h post-transfection, samples from the cell culture fluid are taken and secretion of the reporter-protein ssHRP is detected by incubation of clarified cell supernatant with TMB reagent (BD Biosciences, Pharmingen). After 3 min, the reaction is stopped and absorbance is measured with an ELISA reader (Spectra Rainbow Thermo) at 450 nm to determine ssHRP titers. To furthermore analyse specific productivities, cells are trypsinized after the last measurement, counted using a CASY® cell counter (Schaerfe System) and the specific productivity is calculated by dividing ssHRP titer by total cell number.

### EXAMPLES

### Example 1

### Sly1 and Munc18c are Localized Along the Secretory Pathway in HEK-293

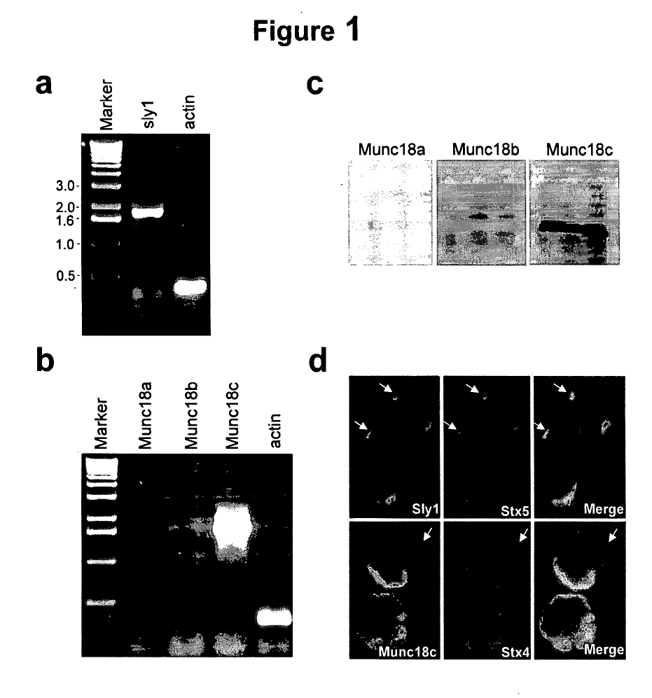

We use RT-PCR-based analysis to profile expression of the SM proteins Sly1 and the isoforms of Munc18 (a, b, c) in HEK-293. As shown in FIGS. 1a and 1b, sly1 (NM_{—}016160) and munc18c (NM_{—}007269) are expressed at high and muc18b (NM_{—}006949) at trace levels while no transcripts of the neuron-specific munc18a (NM_{—}003165) can be detected. The SM protein profiles are confirmed by Western blot (FIG. 1c). Intracellular localization of Sly1 and the major Munc18 isoform, Munc18c, are analyzed by co-expressing YFP-Sly1 (pRP32) and CFP-Syntaxin5 (pRP40), or YFP-Munc18c (pRP23) and CFP-Syntaxin4 (pRP29) in HEK-293. Syntaxin5 is a Sly1-binding SNARE localized at the Golgi apparatus and Syntaxin4 is a Munc 18c-interacting SNARE bound to the plasma membrane. Confocal microscopy shows that Sly1 exhibits a very compact perinuclear co-localization with Syntaxin5 at the Golgi apparatus and the plasma membrane co-stains for Munc18c and Syntaxin4 (FIG. 1d). These results demonstrate that Sly1 and Munc18c are expressed in HEK-293 and localized to the Golgi apparatus and plasma membrane which is consistent with their roles in two distinct fusion steps at the respective organelles (Jahn et al., 2003).

### Example 2

### Sly1 and Munc18 Regulate Protein Secretion

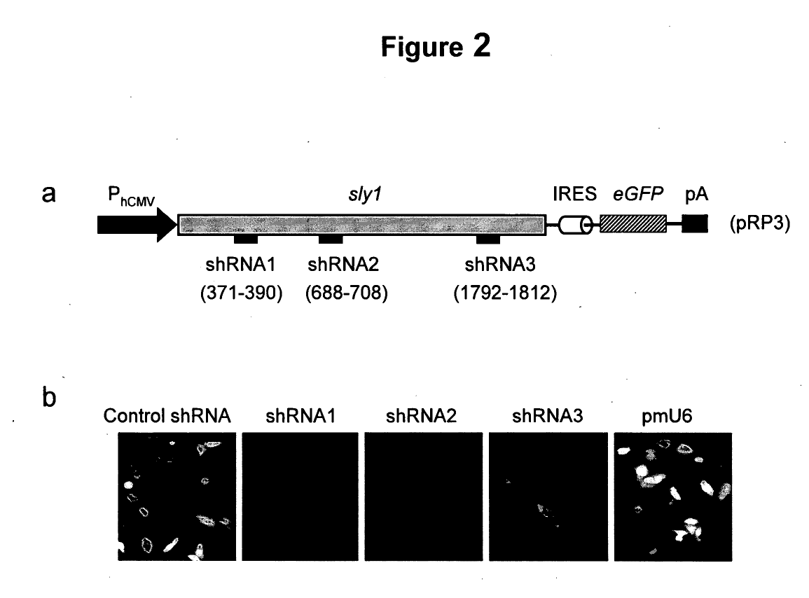

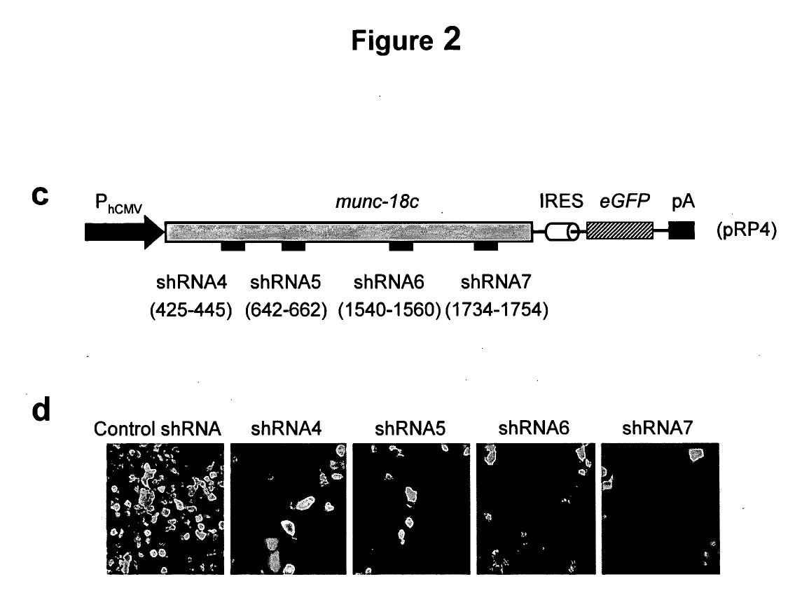

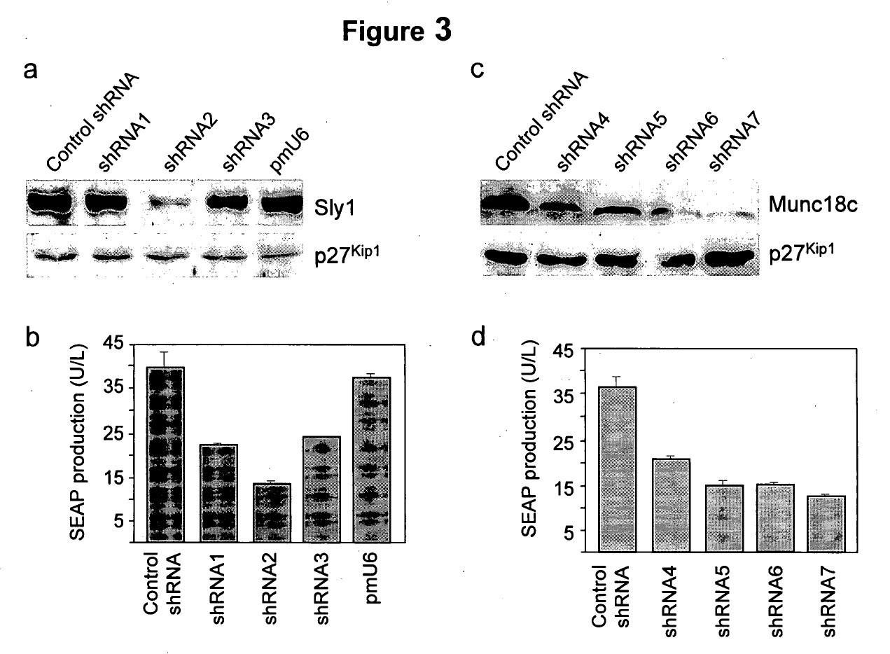

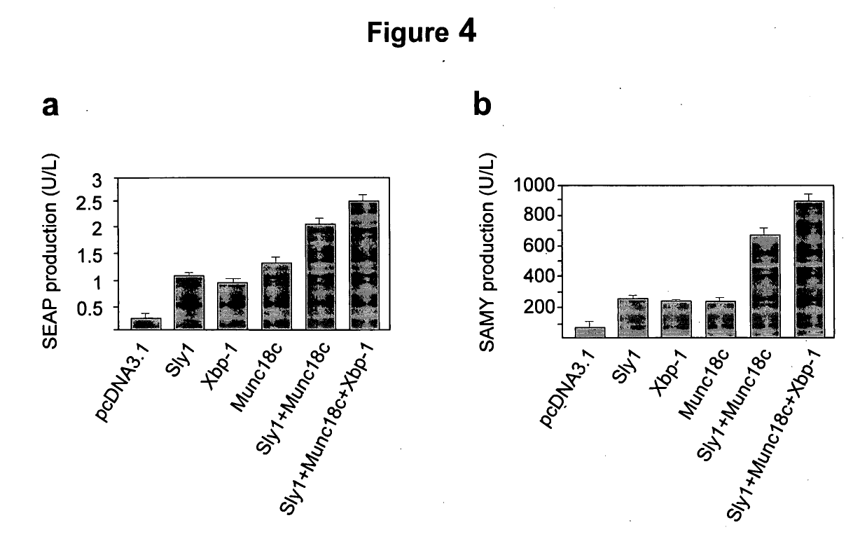

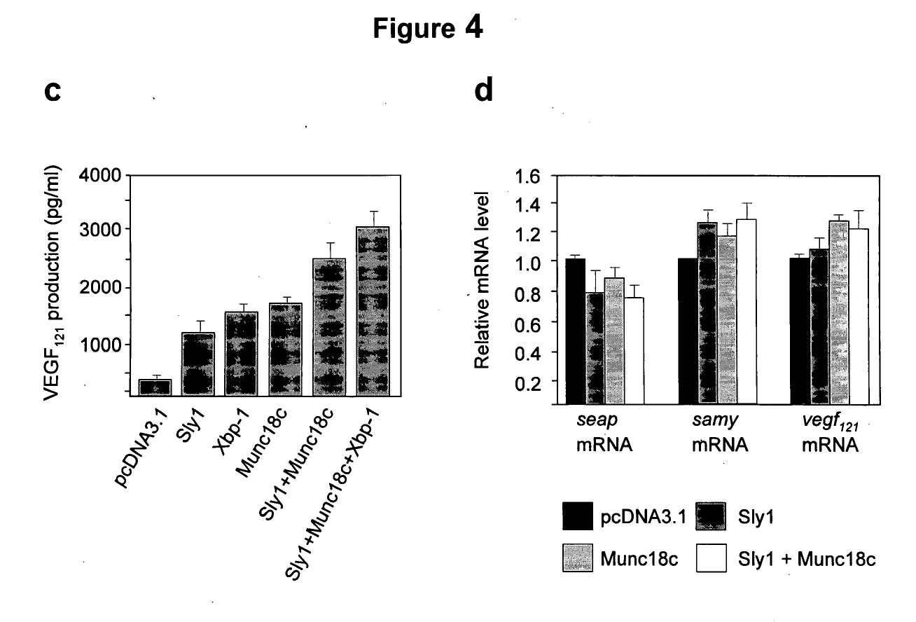

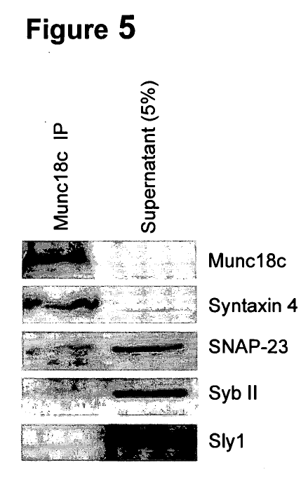

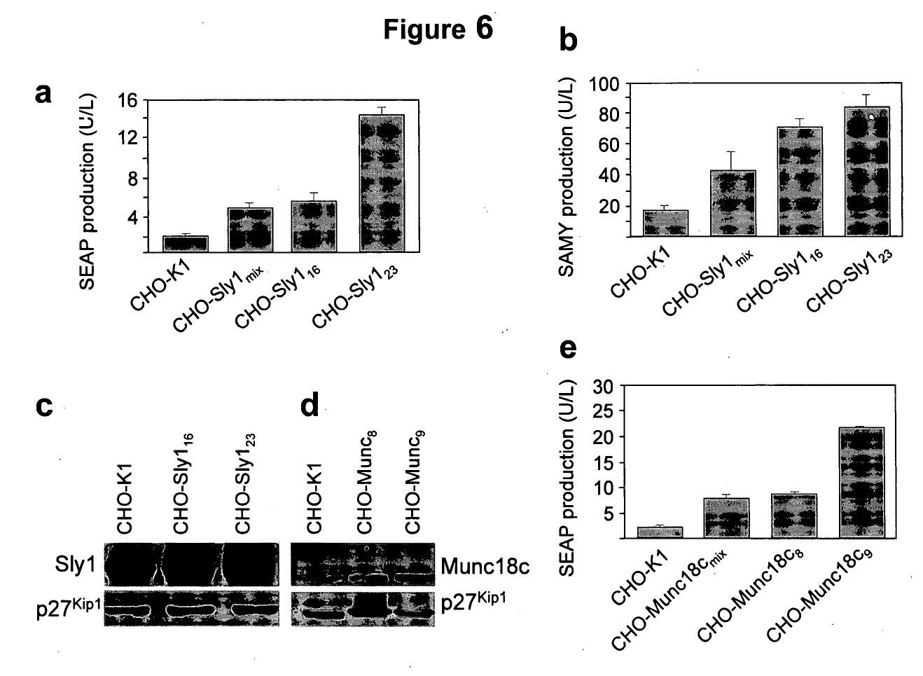

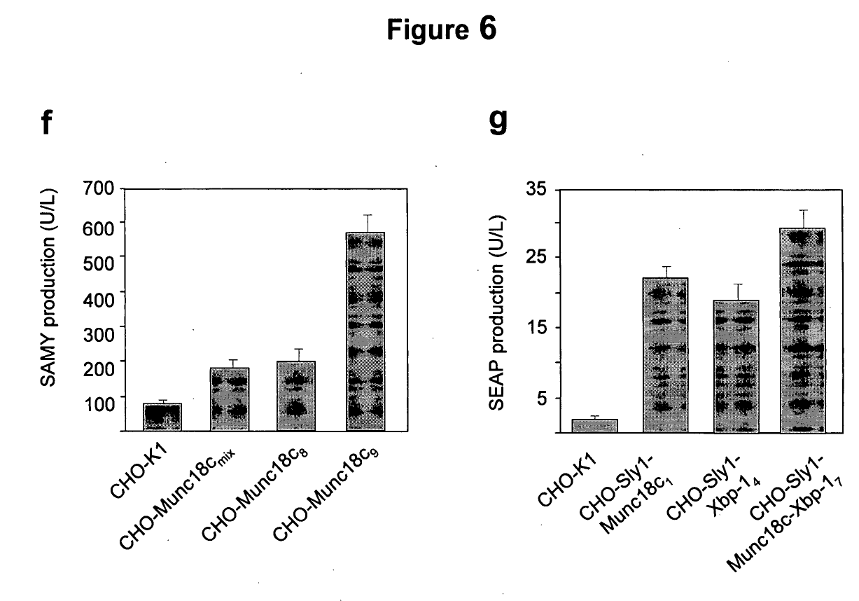

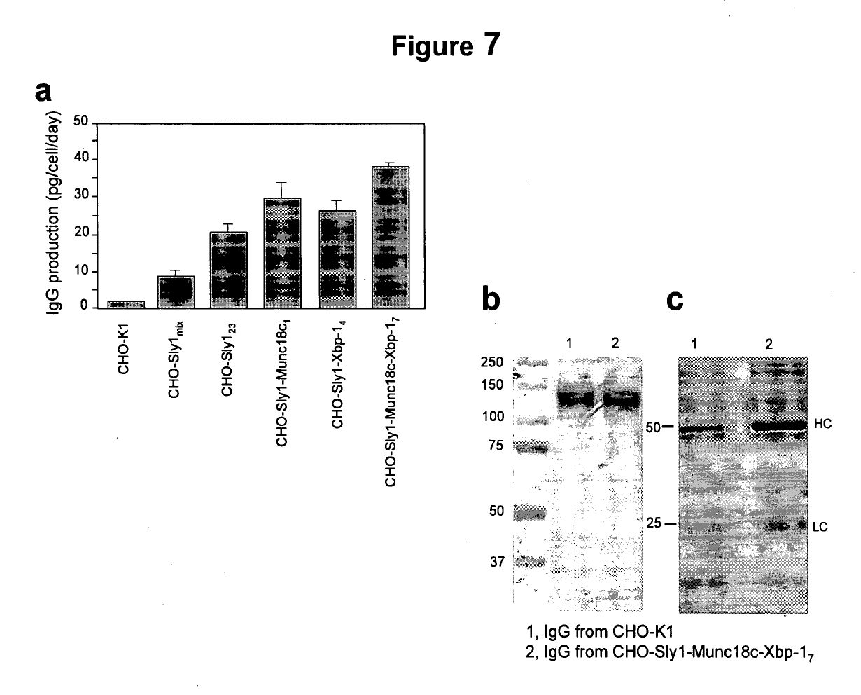

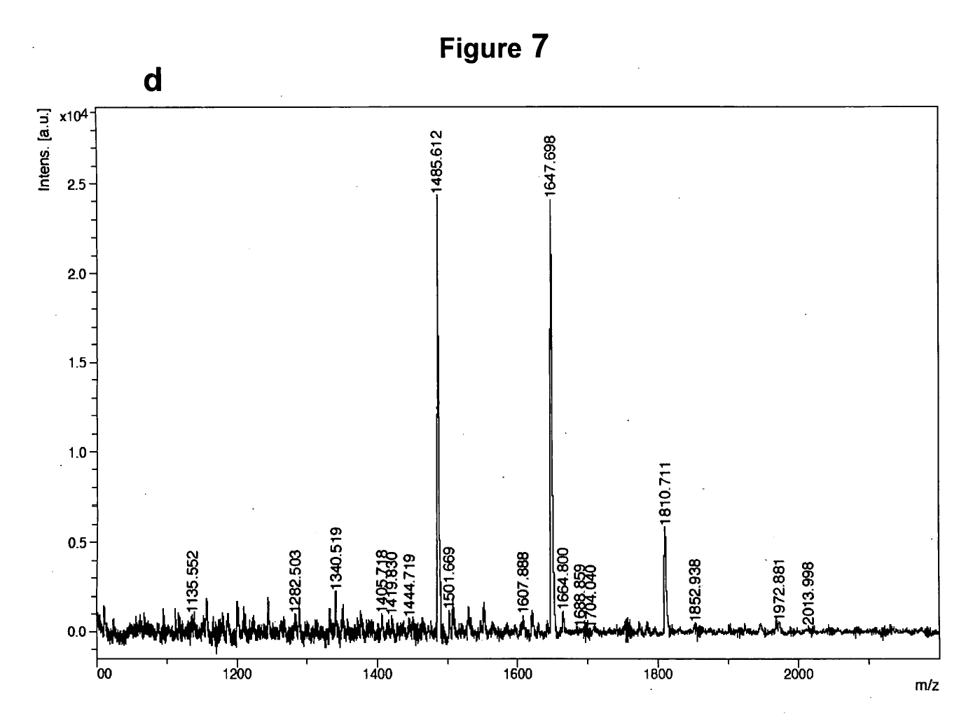

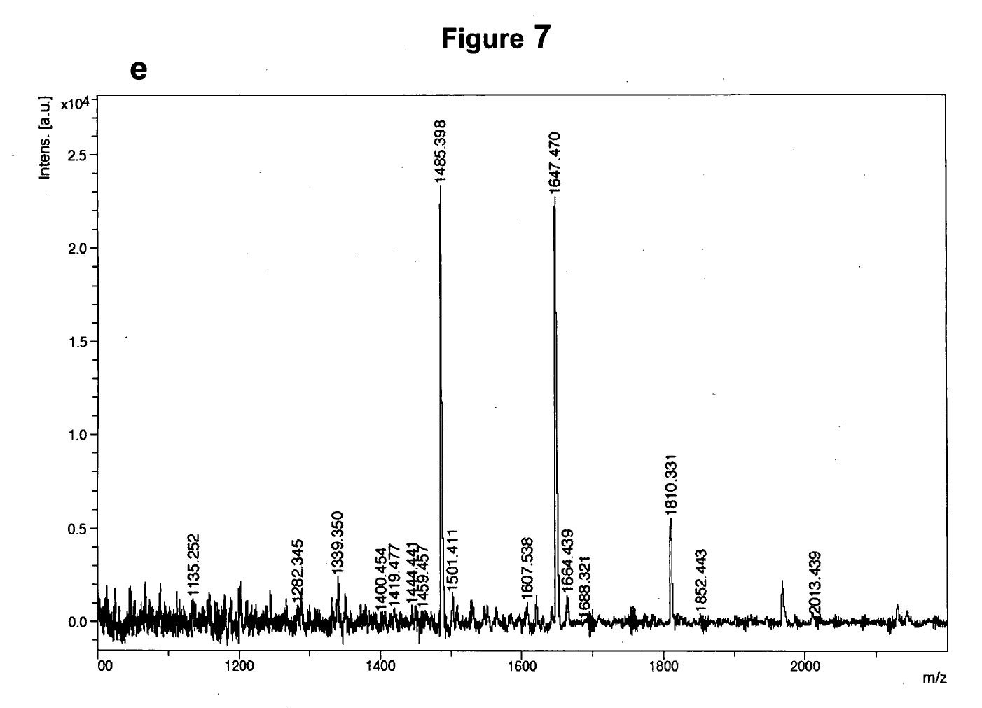

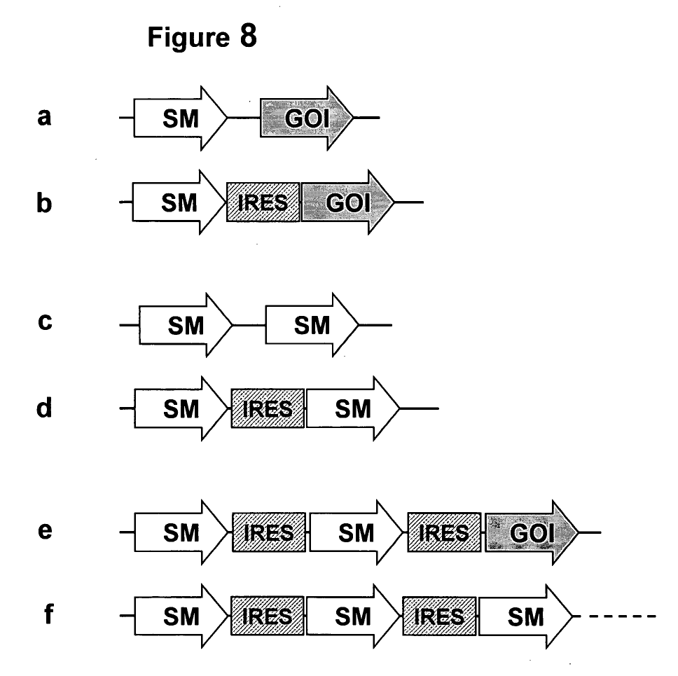

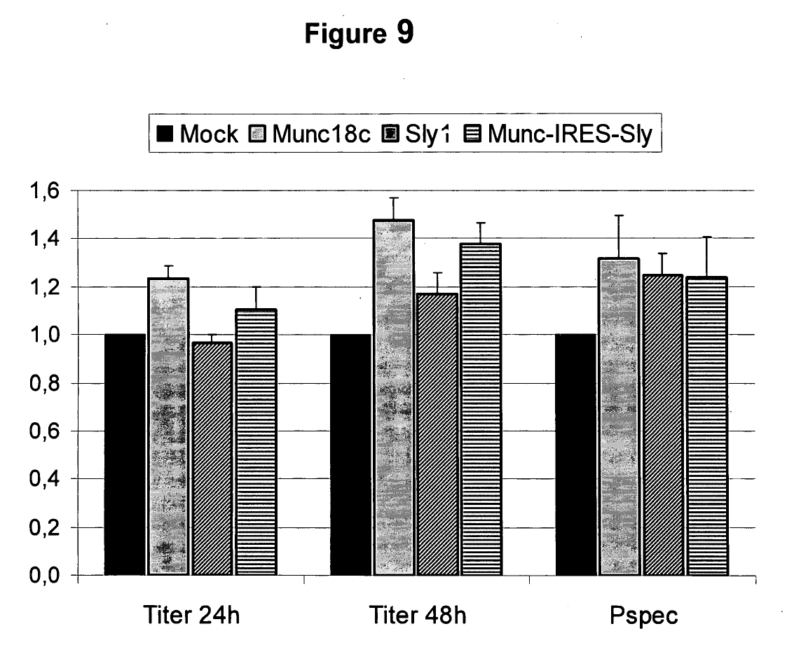

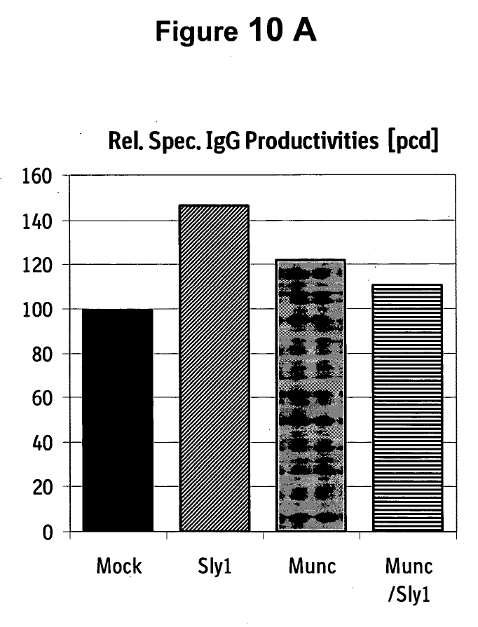

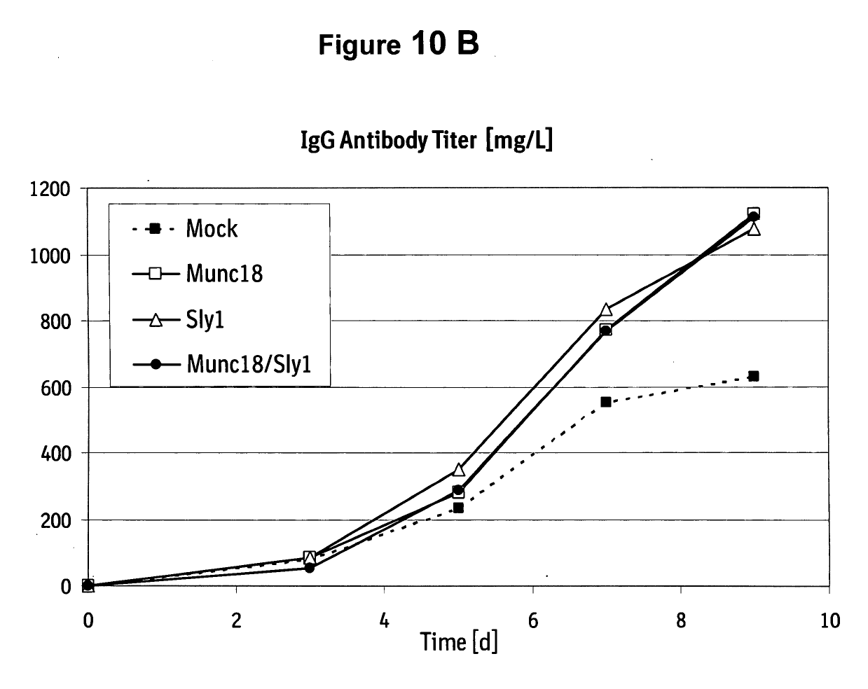

SM proteins are known to control vesicle fusion essential for the intracellular protein traffic but their role for protein secretion remains elusive. To characterize the impact of Sly1 and Munc18 on overall exocytosis, we design shRNAs specific for these SM proteins. Knockdown of Sly1 and Munc 18c is demonstrated by fluorescence microscopy of cells co-transfected with dicistronic Sly1-(pRP3; P_{hCMV}-Sly1-IRES-eGFP-pA) and Munc18c-(pRP4; P_{hCMV}-munc18c-IRES-eGFP-pA) encoding reporter constructs and specific as well as non-specific control shRNAs (FIG. 2). The capacity of individual shRNAs to knockdown endogenous Sly1 and Munc18c expression is confirmed in HEK-293 to reach up to 70% (FIGS. 3a and 3c). To analyze the impact of Sly1 and Munc18c knockdown on the overall protein secretion capacity of mammalian cells we co-transfected pSEAP2-control and pRP5 (shRNA_{sly1}_{_{—}}₁), pRP6 (shRNA_{sly1}_{_{—}}₂), pRP7 (shRNA_{sly1}_{_{—}}₃), or pRP12 (shRNA_{munc18c}_{_{—}}₁), pRP14 (shRNA_{munc18c}_{_{—}}₂), pRP38 (shRNA_{munc18c}_{_{—}}₃), pRP39 (shRNA_{munc18c}_{_{—}}₄) into HEK-293 and profiled SEAP levels in the culture supernatant. The direct correlation of Sly1 and Munc18c knockdown with a decrease in SEAP production suggests a central role of these SM proteins in the mammalian secretory pathway (FIGS. 3b and 3d).

### Example 3

### Ectopic Expression of Sly1 and Munc18c Increase the Secretory Capacity of Mammalian Cells

Following ectopic expression of Sly1 or Munc18c in CHO-K1 (FIGS. 4a, 4b, 4c) heterologous production of SEAP, SAMY or VEGF₁₂₁ is up to 5-fold increased independent of the promoter used to drive product gene transcription (P_{SV40}, P_{hCMV}, P_{EF1α}). Similar results are also observed when HEK-293 cells are used (data not shown). The boost of heterologous protein production is mediated by a posttranslational mechanism, since the mRNA levels of SEAP, SAMY and VEGF are roughly constant in the presence or absence of elevated Sly1, Munc18c or both (FIG. 4d). Our results contrast sharply with previous studies claiming an inhibitory effect of Munc 18 proteins for the exocytosis in a range of cell types including adipocytes and myocytes (Riento et al., 2000; Kanda et al., 2005; Tellam et al., 1997; Thurmond et al., 1998), and provide the first evidence that both Munc18c and Sly1 promote overall exocytosis.

### Example 4

### Synergistic Effect of SM Proteins and Xbp-1 on the Secretory Pathway

Since Sly1 and Munc18 as well as Xbp-1, which have recently been identified to boost protein secretion by increasing the size of secretory organelles (Tigges and Fussenegger, 2006) have different targets in the secretory pathway they might be able to synergistically enhance protein production. We therefore co-transfect different combinations of Sly1-, Munc18c and Xbp-1-encoding and SEAP-, SAMY and VEGF₁₂₁-containing expression vectors into CHO-K1 and profile reporter protein levels in the culture supernatants. As shown in FIG. 4a, simultaneous overexpression of sly1 and munc18c leads to an 8-fold increase in SEAP production, as compared to the 5-fold by sly1 or munc18c alone. Secretion of SAMY and VEGF₁₂₁ is also increased (FIGS. 4b, 4c). Overexpression of sly1, munc18c and xbp-1 altogether increases secretion of SEAP, SAMY and VEGF by 10-, 12- and 8-fold, respectively (FIGS. 4a, 4b, 4c), clearly demonstrating the existence of a synergistic effect on secretion between Sly1 and Munc18c, and between the two SM proteins and the general organelle-expanding factor Xbp-1.

### Example 5

### SM Proteins Enhance the Secretory Capacity by Stimulating the SNARE-Mediated Trafficking Machinery

Previous studies assigned an inhibitory role for Munc18c in exocytosis, which contrasts the results reported here (Riento et al., 2000; Kanda et al., 2005; Tellam et al., 1997; Thurmond et al., 1998). To provide molecular insight into Munc18c's role in the trafficking machinery, in particular its interaction with exocytic SNARE proteins consisting of syntaxin 4, SNAP-23 and VAMP2, we perform immunoprecipitation experiments. As shown in FIG. 5, Munc18c-specific antibodies quantitatively precipitate the Munc18c along with a significant fraction of syntaxin4, SNAP-23 and VAMP 2, indicating the in vivo association of Munc18c with these SNAREs, which facilitate vesicle-organelle fusion in the secretory pathway (Peng and Gallwitz, 2002; Shen et al., 2007; Scott et al., 2004). This finding highlights that, similar to Sly1, which binds to the fully assembled SNARE complexes and facilitates fusion the Golgi apparatus, Munc18c directly interacts with SNARE complexes as well, suggesting a conserved mechanism of action by promoting the SNARE-mediated trafficking machinery.

### Example 6

### SM Protein-Based Engineering of Mammalian Cells for Increased Secretory Capacity in Mammalian Cells

The positive effect of Sly1 and Munc18c expression on the secretory capacity of mammalian cells points to a novel, post-translational approach to engineer mammalian production cell lines for increased secretion. We therefore generate stable CHO-K1-derived cell lines engineered for constitutive expression of either sly1 (CHO-Sly1₁₆ and CHO-Sly1₂₃) or munc18c (CHO-Munc18c₈ and CHO-Munc18c₉). CHO-Sly1₁₆ and CHO-Sly1₂₃ stimulate SEAP secretion by a factor of 4- and 8-fold (FIG. 6a) and SAMY production 4- and 5-fold (FIG. 6b). Interestingly, CHO-Sly1₂₃ producing more SEAP also shows higher Sly1 levels suggesting a positive correlation of SM and product proteins (FIG. 6c). Similarly, cells transgenic for constitutive munc18c expression (CHO-Munc18c₉) produce 9- and 6.5-fold more SEAP and SAMY (FIGS. 6e and 6f) and CHO-Munc18₉ producing more SEAP also shows higher Munc18c levels (FIG. 6d). The stable cell lines CHO-Sly1-Munc18c₁, double-transgenic for constitutive Sly1 and Munc18c expression and CHO-Sly1-Munc18c-Xbp-1₇, triple-transgenic for constitutive Sly1, Munc18c and Xbp-1 expression show 13- and 16-fold higher SEAP production compared to parental CHO-K1 (FIG. 6g).

### Example 7

### SM Protein-Based Secretion Engineering Increases Specific Antibody Productivity of Production Cell Lines

In order to validate SM protein-based secretion engineering in a prototype biopharmaceutical manufacturing scenario we express monoclonal anti-human CD20 IgG1 known as Rituximab in CHO-Sly1₁₆ and CHO-Sly1₂₃ (up to 10-fold increase), in CHO-Sly1-Munc18c₁ (up to 15-fold increase) and in CHO-Sly1-Xbp-1₄ (up to 13-fold increase) and in CHO-Sly1-Munc18c-Xbp-1₇ (up to 19-fold increase) (FIG. 7a). When producing Rituximab in CHO-Sly1-Munc18c-Xbp-1₇ ad hoc production levels of up to 40 pg/cell/day can be reached, which corresponds to a near 20-fold increase compared to an isogenic control cell line (FIG. 7a). SDS-PAGE analysis indicate that the antibodies produced by CHO-Sly1-Munc18c-Xbp-1₇ and wild-type CHO-K1 cells are structurally intact and indistinguishable from each other (FIGS. 7b, 7c). Maldi-TOF-based Glycoprofiling of N-linked Fc oligosaccharides from Rituximab produced in CHO-Sly1-Munc18c-Xbp-1₇ reveals no difference compared to native production cell lines indicating that SM/Xbp-1-based secretion engineering is not compromising the product quality (FIGS. 7d and 7e).

### Example 8

### SM Protein-Based Secretion Engineering Increases Total Antibody Yield in Production Processes

a) To test whether heterologous expression of SM proteins can also be used to enhance therapeutic protein secretion under conditions relevant for industrial manufacturing, an antibody producing CHO cell line (CHO DG44) secreting humanised anti-CD44v6 IgG antibody BIWA 4 is stably transfected with an empty vector (MOCK control) or expression constructs encoding Sly1 (SEQ ID NO. 41) or Munc-18 (SEQ ID NO. 39) or both proteins as a bi-cistronic expression unit. The cells are then subjected to selection to obtain stable cell pools. During six subsequent passages, supernatant is taken from seed-stock cultures of all stable cell pools, the MCP-1 titer is determined by ELISA and divided by the mean number of cells to calculate the specific productivity. In all cells expressing either of the SM proteins, IgG expression is significantly enhanced compared to MOCK or untransfected cells, whereby the highest values are seen in the cell pools simultaneously expressing both SM proteins.

Similar results can be obtained if the stable transfectants are subjected to batch or fed-batch fermentations. Total cell numbers and cell viabilities are measured daily and at days 3, 5, 7, 9 and 11, samples are taken from the cell culture fluid to determine the IgG titer and the specific productivity (FIGS. 10A,B). Under these conditions, the SM protein transgenic cells show similar growth properties compared to the MOCK controls and the un-transfected parental cell line. However compared to MOCK controls, the specific IgG productivities are significantly increased (up to 50% higher) in cells expressing Sly1 or Munc-18 or both SM proteins simultaneously (FIG. 10A), resulting in a clear increase in monoclonal antibody titers in the production process (FIG. 10B.

Taken together, this data demonstrate the applicability of SM protein-based cell engineering approaches to enhance therapeutic protein production in multiple culture formats, including serial cultures, bioreactor batch and fed batch cultures.

b) CHO host cells (CHO DG44) are first transfected with vectors encoding Sly1 (SEQ ID NO. 41) or Munc-18 (SEQ ID NO. 39) or both proteins together. Cells are subjected to selection pressure and cell lines are picked that demonstrate heterologous expression of the SM proteins. Subsequently these cell lines and in parallel CHO DG 44 wild type cells are transfected with expression constructs encoding a human monoclonal IgG-type antibody as the gene of interest. After a second round of selection, supernatant is taken from seed-stock cultures of all stable cell pools over a period of six subsequent passages, the IgG titer is determined by ELISA and divided by the mean number of cells to calculate the specific productivity.

The highest values are seen in the cell pools harbouring both SM proteins, followed by those expressing either Sly1 or Munc-18 alone, which still produce significantly higher antibody titers compared to CHO DG-44 cells that express neither of the SM proteins. Similar results can be obtained if the stable transfectants are subjected to batch or fed-batch fermentations. In each of these settings, overexpression of both SM proteins together leads to a significant increase in both, antibody titers and specific productivities. This indicates that heterologous expression of Sly1 or Munc-18 alone is sufficient to enhance therapeutic antibody secretion. Additionally, heterologous expression of both proteins in combination unitedly increase overall exocytosis in a synergistic fashion in transient as well as stably transfected cell lines.

### Example 9

### Overexpression of SM Proteins Increases Biopharmaceutical Protein Production of Fibroblast Activation Protein Alpha (FAP)

(a) A human fibrosarcoma cell line (HT1080, ATCC CCL-121) expressing the transmembrane gelatinase fibroblast activation protein alpha (FAP) is transfected with an empty vector (MOCK control) or expression constructs encoding Sly1 (SEQ ID NO. 41) or Munc-18 (SEQ ID NO. 39) or both proteins as a bi-cistronic expression unit. The cells are then subjected to selection to obtain stable cell pools. From seed-stock cultures of these pools, cells are harvested and either fixed for determination of FAP surface expression by FACS or cell lysates are prepared for Western blotting using anti-FAP antibodies. Compared to MOCK cells, the amount of FAP on the cell surface is significantly increased in all cells expressing SM proteins and the expression is highest in cells expressing both, Sly1 and Munc-18. This results indicate that both SM proteins act synergistically to enhance the production and transport capacity of cells for a cell-surface transmembrane protein.

b) Human HT1080 or HEK293 cells are first transfected with vectors encoding Sly1 (SEQ ID NO. 41) or Munc-18 (SEQ ID NO. 39) or both proteins together. Cells are subjected to selection pressure and cell lines are picked that demonstrate heterologous expression of the SM proteins. Subsequently these cell lines and in parallel HT1080 or HEK293 wild type cells are transfected with a vector encoding FAP alpha as the gene of interest. After a second round of selection, cells are taken from cultures of all stable cell pools and the expression level of FAP is determined by FACS or Western blotting. The highest values are seen in the cell pools harbouring both SM proteins, followed by those expressing either Sly1 or Munc-18 alone, which still express significantly higher FAP levels compared to parental cells that express neither of the SM proteins. Similar results can be obtained if the stable transfectants are adapted to growth in suspension and are subjected to batch or fed-batch fermentations. In each of these settings, overexpression of both SM proteins together leads to a significant increase in FAP expression. This indicates that heterologous expression of Sly1 and Munc-18 results in improved production and cell-surface localization of transmembrane proteins, whereby the effect is highest upon heterologous introduction of both proteins in combination.

### Example 10

### Overexpression of SM Proteins Increases Biopharmaceutical Protein Production of Transmembrane Protein Epithelial Growth Factor Receptor (EGFR)

(a) A CHO cell line (e.g. CHO-DG44) expressing transmembrane protein epithelial growth factor receptor (EGFR) is transfected with an empty vector (MOCK control) or expression constructs encoding Sly1 (SEQ ID NO. 41) or Munc-18 (SEQ ID NO. 39) or both proteins as a bi-cistronic expression unit. The cells are then subjected to selection to obtain stable cell pools. From seed-stock cultures of these pools, cells are taken during four subsequent passages and the expression level of EGFR is determined by FACS or Western blotting. Compared to MOCK cells, the amount of EGFR on the cell surface is significantly increased in all cells expressing SM proteins and the expression is highest in cells expressing both, Sly1 and Munc-18. Very similar results can be obtained if the stable transfectants are subjected to batch or fed-batch fermentations. In each of these settings, overexpression of either Sly1 or Munc-18 results in a moderate increase in EGFR expression compared to controls, whereas EGFR levels are significantly increased upon simultaneous overexpression of Sly1 and Munc-18, indicating that both SM proteins act synergistically to enhance the production and transport capacity of cells for a cell-surface transmembrane protein in multiple culture formats, including serial cultures, bioreactor batch and fed batch cultures.

b) CHO host cells (CHO DG44) are first transfected with vectors encoding Sly1 (SEQ ID NO. 41) or Munc-18 (SEQ ID NO. 39) or both proteins together. Cells are subjected to selection pressure and cell lines are picked that demonstrate heterologous expression of the SM proteins. Subsequently these cell lines and in parallel CHO DG 44 wild type cells are transfected with a vector encoding the EGFR as the gene of interest. After a second round of selection, cells are taken from seed-stock cultures of all stable cell pools for six consecutive passages and the expression level of EGFR is determined by FACS or Western blotting. The highest values are seen in the cell pools harbouring both SM proteins, followed by those expressing either Sly1 or Munc-18 alone, which still express significantly higher EGFR levels compared to CHO DG-44 cells that express neither of the SM proteins. Similar results can be obtained if the stable transfectants are subjected to batch or fed-batch fermentations. In each of these settings, overexpression of both SM proteins together leads to a significant increase in EGFR expression. This indicates that heterologous expression of Sly1 and Munc-18 results in improved production and cell-surface localization of transmembrane proteins, whereby the effect is highest upon heterologous introduction of both proteins in combination.

### Example 11

### Overexpression of SM Proteins Increases Biopharmaceutical Protein Production of Monocyte Chemoattractant Protein 1 (MCP-1)

(a) A CHO cell line (CHO DG44) secreting monocyte chemoattractant protein 1 (MCP-1) is transfected with an empty vector (MOCK control) or expression constructs encoding Sly1 (SEQ ID NO. 41) or Munc-18 (SEQ ID NO. 39) or both proteins as a bi-cistronic expression unit. The cells are than subjected to selection to obtain stable cell pools. During six subsequent passages, supernatant is taken from seed-stock cultures of all stable cell pools, the MCP-1 titer is determined by ELISA and divided by the mean number of cells to calculate the specific productivity. In all cells expressing either of the SM proteins, IgG expression is significantly enhanced compared to MOCK or untransfected cells, whereby the highest values are seen in the cell pools simultaneously expressing both SM proteins. Similar results can be obtained if the stable transfectants are subjected to batch or fed-batch fermentations. In each of these settings, overexpression of both SM proteins leads to enhanced MCP-1 secretion, indicating that both SM proteins act synergistically to improve the protein production capacity of cells in multiple culture formats, including serial cultures, bioreactor batch and fed batch cultures.

b) CHO host cells (CHO DG44) are first transfected with vectors encoding Sly1 (SEQ ID NO. 41) or Munc-18 (SEQ ID NO. 39) or both proteins together. Cells are subjected to selection pressure and cell lines are picked that demonstrate heterologous expression of the SM proteins. Subsequently these cell lines and in parallel CHO DG 44 wild type cells are transfected with a vector encoding monocyte chemoattractant protein 1 (MCP-1) as the gene of interest. After a second round of selection, supernatant is taken from seed-stock cultures of all stable cell pools over a period of six subsequent passages, the MCP-1 titer is determined by ELISA and divided by the mean number of cells to calculate the specific productivity.

The highest values are seen in the cell pools harbouring both SM proteins, followed by those expressing either Sly1 or Munc-18 alone, which still produce significantly higher MCP-1 titeres compared to CHO DG-44 cells that express neither of the SM proteins. Similar results can be obtained if the stable transfectants are subjected to batch or fed-batch fermentations. In each of these settings, overexpression of both SM proteins together leads to a significant increase in both, MCP-1 titers and specific productivities. This indicates that heterologous expression of Sly1 or Munc-18 alone is sufficient to enhance MCP-1 secretion. However, heterologous expression of both proteins in combination unitedly increase overall exocytosis in a synergistic fashion in transient as well as stably transfected cell lines.

### Example 12

### SM Proteins Enhance HRP Secretion from Human Cells

To address the question of whether overexpression of SM proteins can also be used to enhance secretory transport in non-rodent, especially human, cells, we make use of a plasmid encoding secreted horseradish peroxidase (ssHRP) which can be used as reporter for constitutive protein secretion.

The human fibrosarcoma cell line (HT1080, ATCC CCL-121) is co-transfected with an expression plasmid encoding ssHRP and either an empty vector (Mock control) or expression constructs encoding Sly1 (SEQ ID NO. 41), Munc18 (SEQ ID NO. 39) or both proteins as a bi-cistronic expression unit. 24 and 48 hours post-transfection, samples from the cell culture supernatant are taken and analysed for peroxidase activity. Following measurement, the cells are typsinized and counted to determine the specific productivity of the cells.

Already after 24 hours, a slight increase in ssHRP secretion compared to control cells can be detected in cells expressing Munc18 or both Munc18 and Sly1 (FIG. 9). At 48 hours post-transfection, all cells expressing SM proteins show enhanced ssHRP titers compared to the mock control (FIG. 9). The highest values are measured in samples from cells transfected with Munc18, which display about 1.4-fold increased HRP activity compared to control samples. Also the specific productivities of cells transfected with either Munc18, Sly1 or both SM proteins are significantly enhanced compared to control cells (FIG. 9).

This confirms that both SM proteins are functionally expressed and enhance protein secretion from human cells.

### REFERENCE LIST

Ansel, H. C. and N. G. Popovish, Pharmaceutical Dosage Forms and Drug Delivery Systems, 5th ed., Lea and Febiger (1990)
Carpp, L. N., Ciufo, L. F., Shanks, S. G., Boyd, A. & Bryant, N. J. The Sec1p/Munc18 protein Vps45p binds its cognate SNARE proteins via two distinct modes. J. Cell Biol. 173, 927-936 (2006).
Carr, C. M., Grote, E., Munson, M., Hughson, F. M. & Novick, P. Sec1p binds to SNARE complexes and concentrates at sites of secretion. J. Cell Biol. 146, 333-344 (1999).
D'Andrea-Merrins, M., Chang, L., Lam, A. D., Ernst, S. A. & Stuenkel, E. L. Munc18c interaction with syntaxin4 monomers and SNARE complex intermediates in GLUT4 vesicle trafficking. J. Biol. Chem. 282, 16553-16566 (2007).
Dulubova, I. et al. A conformational switch in syntaxin during exocytosis: role of munc18. EMBO J. 18, 4372-4382 (1999).
Dulubova, I. et al. Munc18-1 binds directly to the neuronal SNARE complex. Proc. Natl. Acad. Sci. USA. 104, 2697-2702 (2007).
Fallaux, F. J., Bout, A., Van Der Velde, I., Van Den Wollenberg, D. J. M., Hehir, K. M., Keegan, J., Auger, C., Cramer, S. J., Van Ormondt, H., Van Der Eb, A. J., Valerio, D., Hoeben, R. C. New helper cells and matched early region 1-deleted adenovirus vectors prevent generation of replication-competent adenoviruses. Human Gene Therapy, 9 (13), pp. 1909-1917 (1998).
Gallwitz, D. & Jahn, R. The riddle of the Sec1/Munc-18 proteins—new twists added to their interactions with SNAREs. Trends Biochem. Sci. 28, 113-116 (2003).
Hooker, A. D., Green, N. H., Baines, A. J., Bull, A. T., Jenkins, N., Strange, P. G., and James, D. C. (1999). Constraints on the transport and glycosylation of recombinant IFN-gamma in Chinese hamster ovary and insect cells. Biotechnol. Bioeng. 63, 559-572.
Hu, S. H., Latham, C. F., Gee, C. L., James. D. E. & Martin, J. L. Structure of the Munc18c/Syntaxin4 N-peptide complex defines universal features of the N-peptide binding mode of Sec1/Munc18 proteins. Proc Natl Acad Sci USA. 104, 8773-8778 (2007).
Iwakoshi, N. N., Lee, A. H., and Glimcher, L. H. (2003). The X-box binding protein-1 transcription factor is required for plasma cell differentiation and the unfolded protein response. Immunol Rev 194, 29-38.
Jahn, R., Lang, T. & Sudhof, T. C. Membrane fusion. Cell 112, 519-533 (2003).
Kanda, H. et al. Adipocyte from Munc 18c-null mice show increased sensitivity to insulin-stimulated GLUT4 externalization. J Clin Invest. 115, 291-301 (2005).
Kosodo, Y., Noda, Y., Adachi, H. & Yoda, K. Binding of Sly1 to Sed5 enhances formation of the yeast early Golgi SNARE complex. J. Cell Sci. 115, 3683-3691 (2002).
Latham, C. F. et al. Molecular dissection of the Munc18c/syntaxin4 interaction: implications for regulation of membrane trafficking. Traffic 7, 1408-1419 (2006).
Lee, A. H., Chu, G. C., Iwakoshi, N. N., and Glimcher, L. H. (2005). XBP-1 is required for biogenesis of cellular secretory machinery of exocrine glands. EMBO J. 24, 4368-4380.
Li, Y., Gallwitz, D. & Peng, R. Structure-based functional analysis reveals a role for the SM protein Sly1 p in retrograde transport to the endoplasmic reticulum. Mol. Biol. Cell 16, 3951-3962 (2005).
Misura, K. M., Scheller, R. H. & Weis, W. I. Three-dimensional structure of the neuronal-Sec1-syntaxin 1a complex. Nature 404, 355-362 (2000).
Peng, R. & Gallwitz, D. Multiple SNARE interactions of an SM protein: Sed5p/Sly1 p binding is dispensable for transport. EMBO J. 23, 3939-3949 (2004).
Peng, R. & Gallwitz, D. Sly1 protein bound to Golgi syntaxin Sed5p allows assembly and contributes to specificity of SNARE fusion complexes. J. Cell Biol. 157, 645-655 (2002).
Riento, K., Kauppi, M., Keranen, S. & Olkkonen, V. M. Munc18-2, a functional partner of syntaxin 3, controls apical membrane trafficking in epithelial cells. J. Biol. Chem. 275, 13476-13483 (2000).
Scott, B. L. et al. Sec1p directly stimulates SNARE-mediated membrane fusion in vitro. J. Cell Biol. 167, 75-85 (2004).
Shen, J., Tareste, D. C., Paumet, F., Rothman, J. E. & Melia. T. J. Selective activation of cognate SNAREpins by Sec1/Munc18 proteins. Cell 128, 183-195 (2007).
Stanley, P. Glycosylation mutants of animal cells. Annu Rev Genet. 18:525-52 (1984).
Tellam, J. T. et al. Characterization of Munc-18c and syntaxin-4 in 3T3-L1 adipocytes. Putative role in insulin-dependent movement of GLUT-4. J. Biol. Chem. 272, 6179-6186 (1997).
Thurmond, D. C. et al. Regulation of insulin-stimulated GLUT4 translocation by Munc18c in 3T3L1 adipocytes. J. Biol. Chem. 273, 33876-33883 (1998).
Tigges, M. & Fussenegger, M. Xbp1-based engineering of secretory capacity enhances the productivity of Chinese hamster ovary cells. Meta. Engineering. 8, 264-272 (2006).
Togneri, J., Cheng, Y. S., Munson, M., Hughson, F. M. & Carr, C. M. Specific SNARE complex binding mode of the Sec1/Munc-18 protein, Sec1p. Proc. Natl. Acad. Sci. USA 103, 17730-17735 (2006).
Toonen, R. F. & Verhage, M. Vesicle trafficking: pleasure and pain from SM genes. Trends Cell Biol. 13, 177-186 (2003).
Urlaub, G., Kas, E., Carothers, A. M., and Chasin, L. A. (1983). Deletion of the diploid dihydrofolate reductase locus from cultured mammalian cells. Cell 33, 405-412.
Urlaub, G., Mitchell, P. J., Kas, E., Chasin, L. A., Funanage, V. L., Myoda, T. T., and Hamlin, J. (1986). Effect of gamma rays at the dihydrofolate reductase locus: deletions and inversions. Somat. Cell Mol. Genet. 12, 555-566.
Verhage, M. et al. Synaptic assembly of the brain in the absence of neurotransmitter secretion. Science 287, 864-869 (2000).
Voets, T. et al. Munc18-1 promotes large dense-core vesicle docking. Neuron 31, 581-591 (2001).
Wu, M. N., Littleton, J. T., Bhat, M. A., Prokop, A. & Bellen, H. J. ROP, the Drosophila Sec1 homolog, interacts with syntaxin and regulates neurotransmitter release in a dosage-dependent manner. EMBO J. 17, 127-139 (1998).
Yang, B., Steegmaier, M., Gonzalez, L. C. Jr., & Scheller, R. H. nSec1 binds a closed conformation of syntaxin1A. J. Cell Biol. 48, 247-252 (2000).
Zilly, F. E., Sorensen, J. B., Jahn, R. & Lang, T. Munc18-bound syntaxin readily forms SNARE complexes with synaptobrevin in native plasma membranes. PLoS Biol. 4, e330 (2006).

1. A method of producing a heterologous protein of interest in a cell comprising:
a) Increasing the expression of at least one gene encoding a protein from the SEC1/Munc18 group of proteins (SM proteins); and
b) Effecting the expression of said heterologous protein of interest.

2. The method according to claim 1, whereby in step b) the expression of said heterologous protein of interest is increased, preferably in step b) the secretion of said heterologous protein of interest is increased.

3. The method according to claim 1, whereby one gene in step a) encodes one of the three Munc18 isoforms, Munc18a, b or c, preferably Munc18c (SEQ ID NO: 39).

4. The method according to claim 1, whereby one gene in step a) encodes Sly-1 (SEQ ID NO: 41).

5. The method according to claim 1, whereby step a) comprises increasing the expression of at least two genes encoding SM-proteins, whereby said SM proteins are involved in two different steps of vesicle transport.

6. The method according to claim 5, whereby:
a) one gene encodes a SM protein, which regulates the fusion of vesicles with the plasma membrane, and
b) the second gene encodes a SM protein, which regulates the fusion of vesicles with the Golgi complex.

7. The method according to claim 5, whereby the expression of Munc18c (SEQ ID NO: 39) and Sly-1 (SEQ ID NO: 41) is increased.

8. The method according to claim 1, whereby step a) comprises
i) increasing the expression of a first gene encoding a member of the SM protein family,
ii) increasing the expression of a second gene encoding another member of the SM protein family, and
iii) increasing the expression of a third gene encoding XBP-1.

9. The method according to claim 8, whereby the expression of Munc18c (SEQ ID NO: 39), Sly-1 (SEQ ID NO: 41), and XBP-1 (SEQ ID NO: 43) is increased.

10. A method of engineering a cell comprising:
a) introducing into a cell one or more vector systems comprising nucleic acid sequences encoding for at least two polypeptides whereby:
i) at least one first nucleic acid sequence encodes a SM-protein, and
ii) a second nucleic acid sequence encodes a protein of interest,
b) expressing said protein of interest and said at least one SM-protein.

11. The method according to claim 10, whereby the SM-protein is either one of the Munc-18 isoforms, preferably Munc-18c (SEQ ID NO: 39), or Sly-1 (SEQ ID NO: 41).

12. The method according to claim 10, whereby in step a) i) two SM-proteins are used in combination, whereby said SM proteins are involved in two different steps of vesicle transport.

13. The method according to claim 12, whereby:
a) one gene encodes a SM protein, which regulates the fusion of vesicles with the plasma membrane,
b) the second gene encodes a SM protein, which regulates the fusion of vesicles with the Golgi complex.

14. The method according to claim 13, whereby the two SM-proteins used in combination are Munc-18c (SEQ ID NO: 39) and Sly-1 (SEQ ID NO: 41).

15. The method according to claim 10, whereby in step a) i) two SM-proteins are used in combination with XBP-1.

16. The method according to claim 15, whereby the SM proteins are Munc-18c (SEQ ID NO: 39) and Sly-1 (SEQ ID NO: 41) in combination with XBP-1 (SEQ ID NO: 43).

17. The method according to claim 1, whereby said cell is a eukaryotic cell, preferably a vertebrate cell, most preferably a mammalian cell.

18. The method according to claim 1, whereby the protein of interest is a therapeutic protein.

19. The method according to claim 18, whereby the protein of interest is an antibody or antibody fragment.

20. Expression vector comprising expression units encoding at least two polypeptides, whereby
a) at least one polypeptide is a SM-protein, and
b) a second polypeptide is a protein of interest.

21. The expression vector according to claim 20, whereby the protein of interest is a therapeutic protein, preferably an antibody or antibody fragment.

22. The expression vector according to claim 20, whereby the expression units are multicistronic, preferably bicistronic.

23. The expression vector according to claim 20, whereby the SM-protein is one of the Munc-18 isoforms Munc-18 a, b, c, preferably Munc-18c (SEQ ID NO: 39).

24. The expression vector according to claim 20, whereby the SM-protein is Sly-1 (SEQ ID NO: 41).

25. The expression vector according to claim 20, whereby at least two SM-proteins are used in combination.

26. The expression vector according to claim 25, whereby the vector comprises at least one bicistronic expression unit arranged as follows:
a) a gene encoding a SM protein,
b) an IRES element and
c) a second gene encoding a SM protein.

27. The expression vector according to claim 20, whereby at least two SM-proteins are used in combination with XBP-1, preferably Munc-18c (SEQ ID NO: 39) and Sly-1 (SEQ ID NO: 41) in combination with XBP-1 (SEQ ID NO: 43).

28. A cell expressing at least two heterologous genes:
a) at least one gene encoding a SM-protein and
b) another gene encoding a protein of interest.

29. The cell according to claim 28, whereby the protein of interest is a therapeutic protein, preferably an antibody or antibody fragment.

30. The cell according to claim 28, whereby the expression level of the SM protein is significantly above the endogenous level, preferably 10%.

31. The cell according to claim 28 comprising an expression vectors comprising expression units encoding at least two polypeptides, whereby
a) at least one polypeptide is a SM-protein, and
b) a second polypeptide is a protein of interest.

32. The cell according to claim 28, whereby said cell is a eukaryotic cell, preferably a vertebrate cell, most preferably a mammalian cell.

33. The cell according to claim 32, whereby said cell is a CHO cell, preferably a CHO DG44 cell.

34. A protein of interest, preferably an antibody produced by the method according to claim 1.

35. A pharmaceutical composition comprising a compound useful for blocking or reducing the activity or expression of one or several SM-proteins and a pharmaceutically acceptable carrier.

36. The pharmaceutical composition according to claim 35 whereby the compound is a polynucleotide sequence.

37. The pharmaceutical composition according to claim 36 whereby the polynucleotide sequence is shRNA, RNAi, siRNA or antisense-RNA, preferably shRNA.

38. The pharmaceutical composition according to claim 35, whereby the SM-protein is Munc-18c (SEQ ID NO: 39) or Sly-1 (SEQ ID NO: 41) or a combination of the two.

39. Method for identifying a modulator of SM-protein function comprising
a) providing at least one SM-protein, preferably Munc-18c,
b) contacting said SM-protein of step a) with a test agent,
c) determining an effect related to increased or decreased protein secretion or expression of cell-surface proteins.

40. A method for the treatment of cancer, auto-immune diseases and inflammation comprising, administering to a patient in need thereof a therapeutically effective amount of a pharmaceutical composition according to claim 35.

41. A method of inhibiting or reducing the proliferation or migration of a cell comprising contacting said cell with a pharmaceutical composition according to claim 35.

42. Use of a SM-protein or a polynucleotide encoding a SM-protein in an in vitro cell or tissue culture system to increase secretion and/or production of a protein of interest.

43. Diagnostic use of the method claim 1.

44. Diagnostic use of the expression vector of claim 20.

45. Diagnostic use of the cell of claim 28.

46. Diagnostic use of the pharmaceutical composition of claim 35.


SEQUENCE LISTING (60 sequences)

SEQ ID NO: 1 - DNA, 33 bp, Artificial Sequence
  Description of Artificial Sequence Synthetic oligonucleotide
cgcggatccaccatggcggcggcggcggcagcg

SEQ ID NO: 2 - DNA, 36 bp, Artificial Sequence
  Description of Artificial Sequence Synthetic oligonucleotide
ccgctcgagttacttttgtccaagttgtgacaactg

SEQ ID NO: 3 - DNA, 36 bp, Artificial Sequence
  Description of Artificial Sequence Synthetic oligonucleotide
cgcggatccaccatggcgccgccggtggcagagagg

SEQ ID NO: 4 - DNA, 32 bp, Artificial Sequence
  Description of Artificial Sequence Synthetic oligonucleotide
ccctcgagctattcatctttaattaaggagac

SEQ ID NO: 5 - DNA, 27 bp, Artificial Sequence
  Description of Artificial Sequence Synthetic oligonucleotide
ctcagatctgcggcggcggcggcagcg

SEQ ID NO: 6 - DNA, 33 bp, Artificial Sequence
  Description of Artificial Sequence Synthetic oligonucleotide
accgtcgaccttttgtccaagttgtgacaactg

SEQ ID NO: 7 - DNA, 35 bp, Artificial Sequence
  Description of Artificial Sequence Synthetic oligonucleotide
cgcgcggccgcaccatggcggcggcggcggcagcg

SEQ ID NO: 8 - DNA, 36 bp, Artificial Sequence
  Description of Artificial Sequence Synthetic oligonucleotide
ccgggatccttacttttgtccaagttgtgacaactg

SEQ ID NO: 9 - DNA, 27 bp, Artificial Sequence
  Description of Artificial Sequence Synthetic oligonucleotide
cccccgggatggtgagcaagggcgagg

SEQ ID NO: 10 - DNA, 27 bp, Artificial Sequence
  Description of Artificial Sequence Synthetic oligonucleotide
tttctagattacttgtacagctcgtcc

SEQ ID NO: 11 - DNA, 38 bp, Artificial Sequence
  Description of Artificial Sequence Synthetic oligonucleotide
cgcgcggccgcaccatggcgccgccggtggcagagagg

SEQ ID NO: 12 - DNA, 32 bp, Artificial Sequence
  Description of Artificial Sequence Synthetic oligonucleotide
ccggatccctattcatctttaattaaggagac

SEQ ID NO: 13 - DNA, 29 bp, Artificial Sequence
  Description of Artificial Sequence Synthetic oligonucleotide
cccaagctttgcgcgacaggacccacgag

SEQ ID NO: 14 - DNA, 33 bp, Artificial Sequence
  Description of Artificial Sequence Synthetic oligonucleotide
cgcgtcgacttatccaacggttatggtgatgcc

SEQ ID NO: 15 - DNA, 27 bp, Artificial Sequence
  Description of Artificial Sequence Synthetic oligonucleotide
ggaagatctatcccgcggaaacgctac

SEQ ID NO: 16 - DNA, 27 bp, Artificial Sequence
  Description of Artificial Sequence Synthetic primer
cccaagctttcaagcaaggaagaccac

SEQ ID NO: 17 - DNA, 16 bp, Artificial Sequence
  Description of Artificial Sequence Synthetic primer
aggcccgggacaggaa

SEQ ID NO: 18 - DNA, 20 bp, Artificial Sequence
  Description of Artificial Sequence Synthetic primer
gccgtccttgagcacatagc

SEQ ID NO: 19 - DNA, 25 bp, Artificial Sequence
  Description of Artificial Sequence Synthetic primer
aaagctcaatatcttcaagccattc

SEQ ID NO: 20 - DNA, 21 bp, Artificial Sequence
  Description of Artificial Sequence Synthetic primer
aacacgacatcggcgtacact

SEQ ID NO: 21 - DNA, 22 bp, Artificial Sequence
  Description of Artificial Sequence Synthetic primer
cttgctgctctacctccaccat

SEQ ID NO: 22 - DNA, 21 bp, Artificial Sequence
  Description of Artificial Sequence Synthetic primer
tgattctgccctcctccttct

SEQ ID NO: 23 - DNA, 58 bp, Artificial Sequence
  Description of Artificial Sequence Synthetic oligonucleotide
tttggaagtaaactggaagatattttcaagagaaatatcttccagtttacttcttttt

SEQ ID NO: 24 - DNA, 58 bp, Artificial Sequence
  Description of Artificial Sequence Synthetic oligonucleotide
ctagaaaaagaagtaaactggaagatatttctcttgaaaatatcttccagtttacttc

SEQ ID NO: 25 - DNA, 60 bp, Artificial Sequence
  Description of Artificial Sequence Synthetic oligonucleotide
tttggcagtgaaactagacaagaaattcaagagatttcttgtctagtttcactgcttttt

SEQ ID NO: 26 - DNA, 60 bp, Artificial Sequence
  Description of Artificial Sequence Synthetic oligonucleotide
ctagaaaaagcagtgaaactagacaagaaatctcttgaatttcttgtctagtttcactgc

SEQ ID NO: 27 - DNA, 60 bp, Artificial Sequence
  Description of Artificial Sequence Synthetic oligonucleotide
tttgggaggcaactacattgaatatttcaagagaatattcaatgtagttgcctccttttt

SEQ ID NO: 28 - DNA, 60 bp, Artificial Sequence
  Description of Artificial Sequence Synthetic oligonucleotide
ctagaaaaaggaggcaactacattgaatattctcttgaaatattcaatgtagttgcctcc

SEQ ID NO: 29 - DNA, 60 bp, Artificial Sequence
  Description of Artificial Sequence Synthetic oligonucleotide
tttgcacatgaatctcaggtgtatattcaagagatatacacctgagattcatgtgttttt

SEQ ID NO: 30 - DNA, 60 bp, Artificial Sequence
  Description of Artificial Sequence Synthetic oligonucleotide
ctagaaaaacacatgaatctcaggtgtatatctcttgaatatacacctgagattcatgtg

SEQ ID NO: 31 - DNA, 60 bp, Artificial Sequence
  Description of Artificial Sequence Synthetic oligonucleotide
tttggcttgaagactactacaagatttcaagagaatcttgtagtagtcttcaagcttttt

SEQ ID NO: 32 - DNA, 60 bp, Artificial Sequence
  Description of Artificial Sequence Synthetic oligonucleotide
ctagaaaaagcttgaagactactacaagattctcttgaaatcttgtagtagtcttcaagc

SEQ ID NO: 33 - DNA, 60 bp, Artificial Sequence
  Description of Artificial Sequence Synthetic oligonucleotide
tttgcgccagaaacccagagctaatttcaagagaattagctctgggtttctggcgttttt

SEQ ID NO: 34 - DNA, 60 bp, Artificial Sequence
  Description of Artificial Sequence Synthetic oligonucleotide
ctagaaaaacgccagaaacccagagctaattctcttgaaattagctctgggtttctggcg

SEQ ID NO: 35 - DNA, 60 bp, Artificial Sequence
  Description of Artificial Sequence Synthetic oligonucleotide
tttggctgaataaacccaaggataattcaagagattatccttgggtttattcagcttttt

SEQ ID NO: 36 - DNA, 60 bp, Artificial Sequence
  Description of Artificial Sequence Synthetic oligonucleotide
ctagaaaaagctgaataaacccaaggataatctcttgaattatccttgggtttattcagc

SEQ ID NO: 37 - DNA, 60 bp, Artificial Sequence
  Description of Artificial Sequence Synthetic oligonucleotide
tttgcacaagctggagtacaactacttcaagagagtagttgtactccagcttgtgttttt

SEQ ID NO: 38 - DNA, 60 bp, Artificial Sequence
  Description of Artificial Sequence Synthetic oligonucleotide
ctagaaaaacacaagctggagtacaactactctcttgaagtagttgtactccagcttgtg

SEQ ID NO: 39 - protein, 592 aa, Homo sapiens
MAPPVAERGLKSVVWQKIKATVFDDCKKEGEWKIMLLDEFTTKLLASCCKMTDLLEEGITVVENIYKNREPVRQMKALYFITPTSKSVDCFLHDFASKSENKYKAAYIYFTDFCPDNLFNKIKASCSKSIRRCKEINISFIPHESQVYTLDVPDAFYYCYSPDPGNAKGKDAIMETMADQIVTVCATLDENPGVRYKSKPLDNASKLAQLVEKKLEDYYKIDEKSLIKGKTHSQLLIIDRGFDPVSTVLHELTFQAMAYDLLPIENDTYKYKTDGKEKEAILEEEDDLWVRIRHRHIAVVLEEIPKLMKEISSTKKATEGKTSLSALTQLMKKMPHFRKQITKQVVHLNLAEDCMNKFKLNIEKLCKTEQDLALGTDAEGQKVKDSMRVLLPVLLNKNHDNCDKIRAILLYIFSINGTTEENLDRLIQNVKIENESDMIRNWSYLGVPIVPQSQQGKPLRKDRSAEETFQLSRWTPFIKDIMEDAIDNRLDSKEWPYCSQCPAVWNGSGAVSARQKPRANYLEDRKNGSKLIVFVIGGITYSEVRCAYEVSQAHKSCEVIIGSTHVLTPKKLLDDIKMLNKPKDKVSLIKDE

SEQ ID NO: 40 - RNA, 2522 nt, Homo sapiens
accccaacgccgcuucugcggccaaaguagguugggaguggaaggugguggcugcugcuccgcagugucgggaagauggcgccgccgguggcagagagggggcuaaagagcgucguguggcagaagauaaaagcaacaguguuugaugacugcaagaaagaaggcgaauggaagauaaugcuuuuagaugaauuuaccacuaagcuuuuggcaucguguugcaaaaugacagaucuucuagaagaagguauuacuguuguagagaauauuuauaagaaccgugaaccugucagacaaaugaaagcucuuuauuucaucacuccgacaucaaagucuguagauuguuucuuacaugauuuugcaaguaaaucggagaacaaguauaaagcagcauauauuuacuucacugacuuuugcccugauaaucucuuuaacaaaauuaaggcuucuugcuccaagucaauaagaagauguaaagaaauaaauauuuccuucauuccacaugaaucucagguguauacucuugauguaccagaugcauucuauuacuguuauaguccagacccugguaaugcaaagggaaaagaugccauuauggaaacaauggcugaccagauaguuacagugugugccaccuuggaugaaaaucccggaguaagauauaaaaguaaaccucuagauaaugccaguaagcuugcacagcuuguugaaaaaaagcuugaagacuacuacaagauugaugaaaagagccuaauaaaggguaaaacucauucacagcucuuaauaauugaucguggcuuugauccuguguccacuguccugcaugaacugaccuuucaggcaauggcauaugaucuacuaccaauugagaaugauacauacaaauauaaaacagauggaaaagaaaaggaggccauccuugaagaagaagaugaccucuggguuagaauucgacaucgacauauugcgguuguguuagaggaaauucccaagcuuaugaaagaaauuucaucaacaaagaaagcaacagaaggaaagacaucacuuagugcucuuacccagcugaugaaaaagaugccccauuuccgaaaacagauuacuaagcaaguuguccaucuuaacuuagcagaagauugcaugaauaaguucaagcuuaauauagaaaagcucugcaaaacugaacaggaccuggcacuuggaacugaugcagaaggacagaaggugaaagauuccaugcgaguacuccuuccaguucuacucaacaaaaaucaugauaauugugauaaaauaagagcaauucuacuuuauaucuucaguauuaauggaacuacggaagaaaauuuggacagguugauccagaauguaaagauagaaaaugagagugacaugauucguaacuggaguuaccuugguguucccauuguuccccaaucucaacaaggcaaaccguuaagaaaggaucggucugcagaagaaacuuuucagcucucucgguggacaccuuuuaucaaagauauuauggaggaugcuauugauaauagauuagauucaaaagaauggccauauuguucccaguguccagcaguauggaaugguucaggagcuguaagugcucgccagaaacccagagcuaauuauuuagaagaccgaaaaaaugggucaaagcugauuguuuuuguaauuggagggaucacauacucugaagugcguugugcuuaugaaguuucucaggcacauaaauccugugaaguuauuauugguucuacacauguuuuaacacccaaaaagcuguuggaugauauaaagaugcugaauaaacccaaggauaaagucuccuuaauuaaagaugaauagcauuucuuuuuggaggguuuagagauucuuacuaauauguugaacuaaaauagaaagaaaauguugcugucauguaauuuaaacaauguaaauauuuuauggaauaauggcuuuucaaauacauuucuuaaggaacuguuuaugauuauuacuggauuugucauuuuugauaauuuaaauauugcugcugcuuuguagaugaugagaagaaauguuaaagugcuuucuaaaaggaaauuuuuucaccuuuggaggagaauauauuagaguuguggguaauuuuucacagccaccuauguacauacuaauuacccauuggauacuuauaucuaaaagucucaugcugaaguauaguuuuugggaaagaaugauuuuaaauaaagagauuguaaaaguaaaaaacuguaaauguauauguaugauagaauuguuuccucuaaguguaguuuuucuuucaacuaaaauucaguuuauguguaaaauaauucagucauuaauagaaauggagugauuucacaguguguacuguuuugccacauacuucuaaagaacacaauuuuauauaauuuugaaaucauguauguuuaaauuagaaaaccaaaaaucaugaacauucuaagagaaaauaaauauagaauuuaaaaaauuaaaaaaaaaaaaaaaaaaaa

SEQ ID NO: 41 - protein, 642 aa, Homo sapiens
MAAAAAATAAAAASIRERQTVALKRMLNFNVPHIKNSTGEPVWKVLIYDRFGQDIISPLLSVKELRDMGITLHLLLHSDRDPIPDVPAVYFVMPTEENIDRMCQDLRNQLYESYYLNFISAISRSKLEDIANAALAASAVTQVAKVFDQYLNFITLEDDMFVLCNQNKELVSYRAINRPDITDTEMETVMDTIVDSLFCFFVTLGAVPIIRCSRGTAAEMVAVKLDKKLRENLRDARNSLFTGDTLGAGQFSFQRPLLVLVDRNIDLATPLHHTWTYQALVHDVLDFHLNRVNLEESSGVENSPAGARPKRKNKKSYDLTPVDKFWQKHKGSPFPEVAESVQQELESYRAQEDEVKRLKSIMGLEGEDEGAISMLSDNTAKLTSAVSSLPELLEKKRLIDLHTNVATAVLEHIKARKLDVYFEYEEKIMSKTTLDKSLLDIISDPDAGTPEDKMRLFLIYYISTQQAPSEADLEQYKKALTDAGCNLNPLQYIKQWKAFTKMASAPASYGSTTTKPMGLLSRVMNTGSQFVMEGVKNLVLKQQNLPVTRILDNLMEMKSNPETDDYRYFDPKMLRGNDSSVPRNKNPFQEAIVFVVGGGNYIEYQNLVDYIKGKQGKHILYGCSELFNATQFIKQLSQLGQK

SEQ ID NO: 42 - RNA, 2172 nt, Homo sapiens
gggcaguggcucgugggagccaagauggcggcggcggcggcagcgacagcagcagcagcagccaguauucgggaaaggcagacaguggcuuugaagcguauguugaauuucaaugugccucauauuaaaaacagcacaggagaaccaguauggaagguacucauuuaugacagauuuggccaagauauaaucucuccucugcuaucugugaaggagcuaagagacaugggaaucacucugcaucugcuuuuacacucugaucgagauccuauuccagauguuccugcaguauacuuuguaaugccaacugaagaaaauauugacagaaugugccaggaucuucgaaaucaacuauaugaaucauauuauuuaaauuuuauuucugcuauuucaagaaguaaacuggaagauauugcaaaugcagcguuagcagcuagugcaguaacacaaguagccaagguuuuugaccaauaucucaauuuuauuacuuuggaagaugauauguuuguauuauguaaucaaaauaaggagcuuguuucauaucgugccauuaacaggccagauaucacagacacggaaauggaaacuguuauggacacuauaguugacagccucuucugcuuuuuuguuacucugggugcuguuccuauaaucagauguucaagaggaacagcagcagaaaugguagcagugaaacuagacaagaaacuucgagaaaaucuaagagaugcaagaaacagucuuuuuacaggugauacacuuggagcuggccaauucagcuuccagaggcccuuauuaguccuuguugacagaaacauagauuuggcaacuccuuuacaucauacuuggacauaucaagcauuggugcacgauguacuggauuuccauuuaaacaggguuaauuuggaagaaucuucaggaguggaaaacucuccagcuggugcuagaccaaagagaaaaaacaagaagucuuaugauuuaacuccgguugauaaauuuuggcaaaaacauaaaggaaguccauucccagaaguugcagaaucaguucagcaagaacuagaaucuuacagagcacaggaagaugaggucaaacgacuuaaaagcauuaugggacuagaaggggaagaugaaggagccauaaguaugcuuucugacaauaccgcuaagcuaacaucagcuguuaguucuuugccagaacuccuugagaaaaaaagacuuauugaucuccauacaaauguugccacugcuguuuuagaacauauaaaggcaagaaaauuggauguauauuuugaauaugaagaaaaaauaaugagcaaaacuacucuggauaaaucucuucuagauauaauaucagacccugaugcaggaacuccagaagauaaaaugagguuguuucuuaucuauuauauaagcacacagcaagcaccuucugaggcugauuuggagcaauauaaaaaagcuuuaacugaugcaggaugcaaccuuaauccuuuacaauauaucaaacaguggaaggcuuuuaccaagauggccucagcuccggccagcuauggcagcacuaccacuaaaccaaugggucuuuuaucacgagucaugaauacaggaucacaguuugugauggaaggagugaagaaccugguuuugaaacagcaaaaucuaccuguuacucguauuuuggacaaucuuauggagaugaagucaaaccccgaaacugaugacuauagauauuuugaucccaaaaugcugcggggcaaugacagcucaguucccagaaauaaaaauccauuccaagaggccauuguuuuuguggugggaggaggcaacuacauugaauaucagaaucuuguugacuacauaaaggggaaacaaggcaaacacauuuuauauggcugcagugagcuuuuuaaugcuacacaguucauaaaacaguugucacaacuuggacaaaaguaacacagaagaaccuuacuaugauaaucuacuuggaauguggauaaauguaaaaagaagaaaaguuagaagagcaauauguuuccuucucuguaacaguguccuaacagugaaaaucagaguuauuuguuaauuuuuaaggaaauuauauacuuaauauguauugauuaaaagaaacauuucagaaauaaaauuucaacauuguuaaaaaaaaaaaaaaaa

SEQ ID NO: 43 - protein, 376 aa, Homo sapiens
MVVVAAAPNPADGTPKVLLLSGQPASAAGAPAGQALPLMVPAQRGASPEAASGGLPQARKRQRLTHLSPEEKALRRKLKNRVAAQTARDRKKARMSELEQQVVDLEEENQKLLLENQLLREKTHGLVVENQELRQRLGMDALVAEEEAEAKGNEVRPVAGSAESAAGAGPVVTPPEHLPMDSGGIDSSDSESDILLGILDNLDPVMFFKCPSPEPASLEELPEVYPEGPSSLPASLSLSVGTSSAKLEAINELIRFDHIYTKPLVLEIPSETESQANVVVKIEEAPLSPSENDHPEFIVSVKEEPVEDDLVPELGISNLLSSSHCPKPSSCLLDAYSDCGYGGSLSPFSDMSSLLGVNHSWEDTFANELFPQLISV

SEQ ID NO: 44 - RNA, 1131 nt, Homo sapiens
augguggugguggcagccgcgccgaacccggccgacgggaccccuaaaguucugcuucugucggggcagcccgccuccgccgccggagccccggccggccaggcccugccgcucauggugccagcccagagaggggccagcccggaggcagcgagcggggggcugccccaggcgcgcaagcgacagcgccucacgcaccugagccccgaggagaaggcgcugaggaggaaacugaaaaacagaguagcagcucagacugccagagaucgaaagaaggcucgaaugagugagcuggaacagcaagugguagauuuagaagaagagaaccaaaaacuuuugcuagaaaaucagcuuuuacgagagaaaacucauggccuuguaguugagaaccaggaguuaagacagcgcuuggggauggaugcccugguugcugaagaggaggcggaagccaaggggaaugaagugaggccaguggccgggucugcugaguccgcagcaggugcaggcccaguugucaccccuccagaacaucuccccauggauucuggcgguauugacucuucagauucagagucugauauccuguugggcauucuggacaacuuggacccagucauguucuucaaaugcccuuccccagagccugccagccuggaggagcucccagaggucuacccagaaggacccaguuccuuaccagccucccuuucucugucaguggggacgucaucagccaagcuggaagccauuaaugaacuaauucguuuugaccacauauauaccaagccccuagucuuagagauacccucugagacagagagccaagcuaaugugguagugaaaaucgaggaagcaccucucagccccucagagaaugaucacccugaauucauugucucagugaaggaagaaccuguagaagaugaccucguuccggagcuggguaucucaaaucugcuuucauccagccacugcccaaagccaucuuccugccuacuggaugcuuacagugacuguggauacggggguucccuuuccccauucagugacauguccucucugcuugguguaaaccauucuugggaggacacuuuugccaaugaacucuuuccccagcugauuagugucuaa

SEQ ID NO: 45 - RNA, 57 nt, Artificial Sequence
  Description of Artificial Sequence Synthetic oligonucleotide
uuuggaaguaaacuggaagauauuuucaagagaaauaucuuccauuuacuucuuuuu

SEQ ID NO: 46 - RNA, 58 nt, Artificial Sequence
  Description of Artificial Sequence Synthetic oligonucleotide
cuagaaaaagaaguaaacuggaagauauuucucuugaaaauaucuuccaguuuacuuc

SEQ ID NO: 47 - RNA, 60 nt, Artificial Sequence
  Description of Artificial Sequence Synthetic oligonucleotide
uuuggcagugaaacuagacaagaaauucaagagauuucuugucuaguuucacugcuuuuu

SEQ ID NO: 48 - RNA, 60 nt, Artificial Sequence
  Description of Artificial Sequence Synthetic oligonucleotide
cuagaaaaagcagugaaacuagacaagaaaucucuugaauuucuugucuaguuucacugc

SEQ ID NO: 49 - RNA, 60 nt, Artificial Sequence
  Description of Artificial Sequence Synthetic oligonucleotide
uuugggaggcaacuacauugaauauuucaagagaauauucaauguaguugccuccuuuuu

SEQ ID NO: 50 - RNA, 60 nt, Artificial Sequence
  Description of Artificial Sequence Synthetic oligonucleotide
cuagaaaaaggaggcaacuacauugaauauucucuugaaauauucaauguaguugccucc

SEQ ID NO: 51 - RNA, 60 nt, Artificial Sequence
  Description of Artificial Sequence Synthetic oligonucleotide
uuugcacaugaaucucagguguauauucaagagauauacaccugagauucauguguuuuu

SEQ ID NO: 52 - RNA, 60 nt, Artificial Sequence
  Description of Artificial Sequence Synthetic oligonucleotide
cuagaaaaacacaugaaucucagguguauaucucuugaauauacaccugagauucaugug

SEQ ID NO: 53 - RNA, 60 nt, Artificial Sequence
  Description of Artificial Sequence Synthetic oligonucleotide
uuuggcuugaagacuacuacaagauuucaagagaaucuuguaguagucuucaagcuuuuu

SEQ ID NO: 54 - RNA, 60 nt, Artificial Sequence
  Description of Artificial Sequence Synthetic oligonucleotide
cuagaaaaagcuugaagacuacuacaagauucucuugaaaucuuguaguagucuucaagc

SEQ ID NO: 55 - RNA, 60 nt, Artificial Sequence
  Description of Artificial Sequence Synthetic oligonucleotide
uuugcgccagaaacccagagcuaauuucaagagaauuagcucuggguuucuggcguuuuu

SEQ ID NO: 56 - RNA, 60 nt, Artificial Sequence
  Description of Artificial Sequence Synthetic oligonucleotide
cuagaaaaacgccagaaacccagagcuaauucucuugaaauuagcucuggguuucuggcg

SEQ ID NO: 57 - RNA, 60 nt, Artificial Sequence
  Description of Artificial Sequence Synthetic oligonucleotide
uuuggcugaauaaacccaaggauaauucaagagauuauccuuggguuuauucagcuuuuu

SEQ ID NO: 58 - RNA, 60 nt, Artificial Sequence
  Description of Artificial Sequence Synthetic oligonucleotide
cuagaaaaagcugaauaaacccaaggauaaucucuugaauuauccuuggguuuauucagc

SEQ ID NO: 59 - RNA, 60 nt, Artificial Sequence
  Description of Artificial Sequence Synthetic oligonucleotide
uuugcacaagcuggaguacaacuacuucaagagaguaguuguacuccagcuuguguuuuu

SEQ ID NO: 60 - RNA, 60 nt, Artificial Sequence
  Description of Artificial Sequence Synthetic oligonucleotide
cuagaaaaacacaagcuggaguacaacuacucucuugaaguaguuguacuccagcuugug